(19) United States
(12) Patent Application Publication
(10) Pub. No.: US 2025/0204937 A1
(43) Pub. Date: Jun. 26, 2025

(54) ASPIRATION SYSTEMS, METHODS, AND DEVICES

(71) Applicant: WALK VASCULAR, LLC, Irvine, CA (US)

(72) Inventors: Randolf VON OEPEN, Aptos, CA (US); Connie Vien CHENG, San Jose, CA (US); Nora V. LEGARDA, San Jose, CA (US)

(21) Appl. No.: 18/967,093

(22) Filed: Dec. 3, 2024

Related U.S. Application Data
(60) Provisional application No. 63/614,011, filed on Dec. 22, 2023.

(51) Int. Cl. A61B 17/22; A61B 17/3203; A61M 25/00

(52) U.S. Cl. CPC A61B 17/22; A61B 17/32037; A61M 25/0026; A61M 25/0045; A61B 2017/22079; A61M 2025/0046

(57) ABSTRACT

An aspiration catheter comprising an aspiration lumen configured to aspirate thrombus from a body lumen, a supply tube with a supply lumen configured to provide a high pressure fluid injection to the aspiration lumen, and a mount associated within the aspiration lumen and configured to position the supply lumen towards one side of the aspiration lumen to prevent blockage of the aspiration lumen through entanglement of thrombus with the supply tube.

### CROSS-REFERENCE TO RELATED APPLICATIONS

This application claims the benefit of and priority to U.S. Provisional Patent Application Ser. No. 63/614,011 filed Dec. 22, 2023, and entitled “ASPIRATION SYSTEMS, METHODS, AND DEVICES,” which application is expressly incorporated herein by reference in its entirety.

### BACKGROUND OF THE INVENTION

### Technical Field

The present disclosure pertains generally to medical devices and methods of their use. More particularly, the present invention pertains to aspiration and thrombectomy devices and methods of attaching a supply tube of pressurized fluid to an aspiration device.

### Description of the Related Art

Several devices and systems already exist to aid in the removal of thrombotic material. These include simple aspiration tube type devices using vacuum syringes to extract thrombus into the syringe, simple flush-and-aspirate devices, more complex devices with rotating components that pull in, macerate and transport thrombotic material away from the distal tip using a mechanical auger, and systems that use very high pressure to macerate the thrombus and create a venturi effect to flush the macerated material away. All of the devices described above have limitations as a result of individual design characteristics.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Embodiments of the present invention provide systems, methods, and devices for aspirating thrombus or material within a patient's body.

Implementations of the present invention solve one or more problems in the art with systems, methods, and devices for aspirating thrombus or material within a patient's body. For instance, the present invention relates aspiration systems, methods and devices for forming an aspiration catheter and positioning at least one supply tube to provide high pressure fluid for injection into the aspiration lumen.

An embodiment of the present disclosure relates to an aspiration catheter including: an aspiration lumen configured to aspirate thrombus from a body lumen; a supply tube with a supply lumen configured to provide a high pressure fluid injection to the aspiration lumen; and a mount associated within the aspiration lumen and configured to position the supply lumen towards one side of the aspiration lumen to prevent blockage of the aspiration lumen through entanglement of thrombus with the supply tube.

An embodiment of the present disclosure relates to an aspiration catheter including: an elongate shaft configured for placement within a blood vessel; a supply lumen and an aspiration lumen each extending along the elongate shaft, the supply lumen having a proximal end and a distal end, and the aspiration lumen having a proximal end and a distal opening, the supply lumen being disposed externally to the aspiration lumen; and an orifice at or near the distal end of the supply lumen, the orifice being configured to allow injection of pressurized fluid into the aspiration lumen at or near the distal end of the aspiration lumen when the pressurized fluid is delivered through the supply lumen.

An embodiment of the present disclosure relates to an aspiration catheter including: an elongate shaft configured for placement within a blood vessel, the elongate shaft having a distal end with an opening at the distal end; a supply lumen and an aspiration lumen each extending along the elongate shaft, the supply lumen having a proximal end and a distal end, and the aspiration lumen having a proximal end and a distal opening, the supply lumen being disposed within a supply tube and the aspiration lumen being formed from a shaft including a plurality of shaft body segments and a plurality of supply tube mount segments, each supply tube mount segment including a mount orifice configured to slidably receive the supply tube; and an orifice near the distal end of the supply lumen, the orifice being configured to allow injection of pressurized fluid into the aspiration lumen at or near the distal end of the aspiration lumen when the pressurized fluid is delivered through the supply lumen.

An embodiment of the present disclosure relates to an aspiration catheter including: an elongate shaft configured for placement within a blood vessel, the elongate shaft having a distal end with an opening at the distal end; a supply lumen and an aspiration lumen each extending along an interior of the elongate shaft, coextensive with the elongate shaft, the supply lumen having a proximal end and a distal end, and the aspiration lumen having a proximal end and a distal opening, the supply lumen being disposed within a supply tube disposed within a plurality of mounts disposed within the aspiration lumen, the plurality of mounts being configured to position the supply lumen towards one side of the aspiration lumen to prevent blockage of the aspiration lumen through entanglement of thrombus with the supply tube; and an orifice near the distal end of the supply lumen, the orifice being configured to allow injection of pressurized fluid into the aspiration lumen at or near the distal end of the aspiration lumen when the pressurized fluid is pumped through the supply lumen.

An embodiment of the present disclosure relates to a system for aspirating thrombus, including: an aspiration catheter including: an elongate shaft configured for placement within a blood vessel of a subject; a supply lumen and an aspiration lumen each extending along an interior of the elongate shaft, the supply lumen being disposed within a supply tube disposed within a plurality of mounts disposed within the aspiration lumen, the plurality of mounts being configured to position the supply lumen towards one side of the aspiration lumen to prevent blockage of the aspiration lumen through entanglement of thrombus with the supply tube; and an orifice at or near a distal end of the supply lumen, the orifice being configured to allow injection of pressurized fluid into the aspiration lumen at or near the distal end of the aspiration lumen when the pressurized fluid is caused or allowed to flow through the supply lumen; a tubing set including a first lumen having a distal end configured to couple to the aspiration lumen of the aspiration catheter and a proximal end configured to couple to a vacuum source, and a second lumen having a distal end configured to couple to the supply lumen of the aspiration catheter and a proximal end configured to couple to a first fluid source; and a pressurization element configured to couple to the tubing set and further configured to pressurize fluid from the first fluid source or allow pressurized fluid from the first fluid source to be delivered to the supply lumen, such that the pressurized fluid is capable of flowing through the supply lumen from the proximal end of the supply lumen to the orifice at or near the distal end of the supply lumen.

An embodiment of the present disclosure relates to a system for aspirating thrombus, including: an aspiration catheter including: an elongate shaft configured for placement within a blood vessel of a subject; a supply lumen and an aspiration lumen each extending along an interior of the elongate shaft, the supply lumen being disposed within a supply tube disposed within a plurality of mounts disposed within the aspiration lumen, the plurality of mounts being configured to position the supply lumen towards one side of the aspiration lumen to prevent blockage of the aspiration lumen through entanglement of thrombus with the supply tube; and an orifice at or near a distal end of the supply lumen, the orifice being configured to allow injection of pressurized fluid into the aspiration lumen at or near the distal end of the aspiration lumen when the pressurized fluid is caused or allowed to flow through the supply lumen; a tubing set including a first lumen having a distal end configured to couple to the aspiration lumen of the aspiration catheter and a proximal end configured to couple to a vacuum source, and a second lumen having a distal end configured to couple to the supply lumen of the aspiration catheter and a proximal end configured to couple to a first fluid source; and a pressurization element configured to couple to the tubing set and further configured to pressurize fluid from the first fluid source or allow pressurized fluid from the first fluid source to be delivered to the supply lumen, such that the pressurized fluid is capable of flowing through the supply lumen from the proximal end of the supply lumen to the orifice at or near the distal end of the supply lumen.

An embodiment of the present disclosure relates to a method for forming an aspiration catheter, the method including: positioning a supply tube mount within a support mandrel; disposing the support mandrel within an aspiration lumen of a shaft body; applying a force from a pusher to the support mandrel to position the supply tube mount against an inner surface of the aspiration lumen; and following coupling the supply tube mount to the shaft body, removing the pusher and the support mandrel.

An embodiment of the present disclosure relates to a method for forming an aspiration catheter, the method including: (a) positioning a first end of a supply tube mount into engagement with a first segment of a shaft body of the aspiration catheter; (b) positioning a second end of the supply tube mount into engagement with a second segment of a shaft body of the aspiration catheter; (c) following aligning the first segment and the second segment, coupling the first segment, the second segment, and the supply tube mount, and (d) repeating steps (a)-(c) until a length of the aspiration catheter is achieved.

An embodiment of the present disclosure relates to a method for forming an aspiration catheter, the method including: positioning a supply tube upon an outer surface of a shaft body of an aspiration catheter to position an orifice of the supply tube distal a distal end of the shaft body, the orifice being in fluid communication with a supply lumen, the orifice being configured to allow injection of pressurized fluid; and mounting a jacket around the supply tube and the shaft body, a distal end of the jacket being distal the orifice, the jacket forming a distal opening of the aspiration catheter.

In any of the described embodiments, the elongate shaft of the aspiration catheter can have an inner diameter of about 0.080 inches (2 mm), or from about 0.080 inches (2 mm) to about 0.28 inches (7 mm), or from about 0.080 inches (2 mm) to about 0.16 inches (4 mm).

In any of the described embodiments, the supply lumen can provide the pressurized fluid at a pressure of about 435 psi (3 MPa) to about 1015 psi (7 MPa), from about 400 psi (2.6 MPa) to about 3,000 psi (20.7 MPa), from about 400 psi (2.6 MPa) to about 2,000 psi (13.8 MPa), or from about 500 psi (3.4 MPa) to about 2,000 psi (13.8 MPa), or from about 600 psi (4.1 MPa) to about 1,750 psi (12.1 MPa). Such pressure may be that measured at a proximal location as delivered to the proximal end of the supply lumen, or as delivered at a distal end, e.g., at the orifice.

In any of the described embodiments, at least one of the orifices associated with the supply lumen can be about 0.05 mm (0.002 inches) by about 0.15 mm (0.006 inches) (e.g., configured as a generally rectangular hole). In some instances, the orifices are configured as a rectangular slot, a circular slot, a tear drop slot, a triangular slot, or any other geometric slot shape. Generally, at least one of the orifices may comprise a generally rectangular or other shaped hole with one or more sides thereof having a length between 0.02 mm (0.0008 inches) and 0.20 mm (0.008 inches). As another example, at least one of the orifices may comprise a generally rectangular hole with each side thereof having a length between 0.02 mm (0.0008 inches) and 0.20 mm (0.008 inches). In some implementations, the total cross-sectional area of all orifices provided in a given supply lumen, or as provided in the aspiration catheter as a whole may be from 0.002 mm² and 0.02 mm², or from 0.003 mm² to about 0.015 mm², or from about 0.005 mm² to about 0.01 mm². In another example, at least one of the orifices may comprise a generally circular hole with a diameter from about 0.03 mm (0.001 inches) to about 0.15 mm (0.006 inches), or from about 0.0508 mm (0.002 inches) to about 0.1016 mm (0.004 inches), or about 0.0787 mm (0.0031 inches).

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is a plan view of exemplary disposable components of a system for aspirating thrombus according to an implementation of the present disclosure.

FIG. 2 is a sectional view of an exemplary distal end of the aspiration catheter of the system for aspirating thrombus of FIG. 1 according to an implementation of the present disclosure.

FIG. 3 is a detail view of an exemplary y-connector of the aspiration catheter of the system for aspirating thrombus of FIG. 1 according to an implementation of the present disclosure.

FIG. 4 is a plan view of exemplary disposable components of a system for aspirating thrombus according to an implementation of the present disclosure.

FIG. 5 is a perspective view of an exemplary system for aspirating thrombus of FIG. 3 according to an implementation of the present disclosure.

FIG. 6 is a perspective view of an exemplary aspiration catheter according to an implementation of the present disclosure.

FIG. 7 is a sectional view of an exemplary aspiration catheter according to an implementation of the present disclosure.

FIG. 8 illustrates the exemplary aspiration catheter within a body lumen aspirating thrombus according to an implementation of the present disclosure.

FIG. 9 illustrates a sectional view of another exemplary aspiration catheter according to an implementation of the present disclosure.

FIG. 10 illustrates a partially exploded view of the another exemplary aspiration catheter of FIG. 9 during manufacturing of the another exemplary aspiration catheter according to an implementation of the present disclosure.

FIGS. 10A-10B illustrate an example of how a flat wire coil may be welded into the inside of a hypotube 417a.

FIG. 11 illustrates a side view of another exemplary aspiration catheter according to an implementation of the present disclosure.

FIG. 12 illustrates an end view of a supply tube mount according to an implementation of the present disclosure.

FIG. 13 illustrates a close-up view of the supply tube mount according to an implementation of the present disclosure.

FIG. 14 illustrates a sectional view of another exemplary aspiration catheter according to an implementation of the present disclosure.

FIG. 15 illustrates a sectional view of another exemplary aspiration catheter according to an implementation of the present disclosure.

FIG. 16 illustrates a portion of a manufacturing process to form the another exemplary aspiration catheter of FIG. 15 according to an implementation of the present disclosure.

FIG. 17 illustrates another portion of a manufacturing process to form the another exemplary aspiration catheter of FIG. 15 according to an implementation of the present disclosure.

FIG. 18 illustrates a sectional view of a support mandrel according to an implementation of the present disclosure.

FIG. 19 illustrates a sectional view of a supply tube mount according to an implementation of the present disclosure.

FIG. 20 illustrates a sectional view of another exemplary aspiration catheter according to an implementation of the present disclosure.

FIG. 21 illustrates a sectional view of another exemplary aspiration catheter according to an implementation of the present disclosure.

FIG. 22 illustrates a sectional view of an exemplary aspiration catheter such as that of FIG. 21 according to an implementation of the present disclosure.

FIG. 23 illustrates a sectional view of another exemplary aspiration catheter similar to that of FIG. 21 according to an implementation of the present disclosure.

FIG. 23A illustrates another exemplary aspiration catheter according to an implementation of the present disclosure.

FIG. 24 illustrates a sectional view of another exemplary aspiration catheter according to an implementation of the present disclosure.

FIG. 25 illustrates a portion of a manufacturing process to form the another exemplary aspiration catheter of FIG. 24 according to an implementation of the present disclosure.

FIG. 26 illustrates another portion of a manufacturing process to form the another exemplary aspiration catheter of FIG. 24 according to an implementation of the present disclosure.

FIG. 27 illustrates a sectional view of a support mandrel according to an implementation of the present disclosure.

FIG. 28 illustrates a sectional view of another exemplary aspiration catheter according to an implementation of the present disclosure.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to manners of mounting a supply tube, with associated supply lumen to limit or prevent blockage of an aspiration lumen of an aspiration catheter. This limits the possibility of engagement or collection of thrombus, clots, or other aspirants between the supply tube and the aspiration catheter thereby reducing the aspiration effectiveness of the aspiration catheter.

While the present disclosure will describe a particular implementation of an aspiration catheter with supply tube mounts or other structures that can be used to position a supply tube of an aspiration system to limit or prevent blockage of an aspiration lumen of an aspiration catheter during aspiration of thrombus, clots, or other aspirants, it should be understood that the devices, systems, and method described herein may be applicable to other uses. Additionally, elements described in relation to any embodiment depicted and/or described herein may be combinable with elements described in relation to any other embodiment depicted and/or described herein.

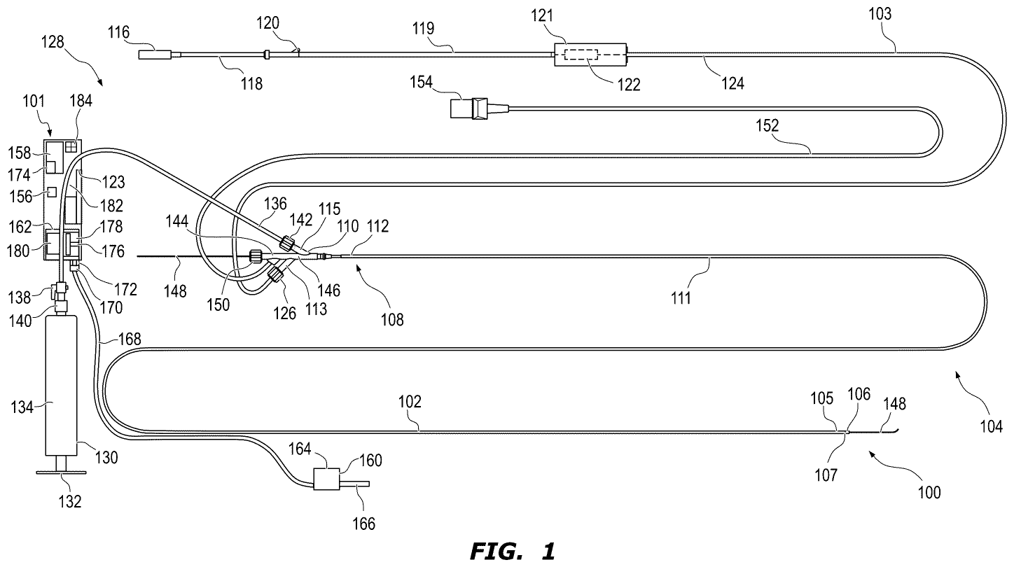

A system 100 for aspirating thrombus is illustrated in FIGS. 1-5. system 100 for aspirating thrombus includes a pump 101, an aspiration catheter 102, and a tubing set 103. The aspiration catheter 102 and the tubing set 103 represent disposable components 104, and the pump 101, and the pump's associated pump base, is a reusable component. It is not necessary to sterilize the pump 101 as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled).

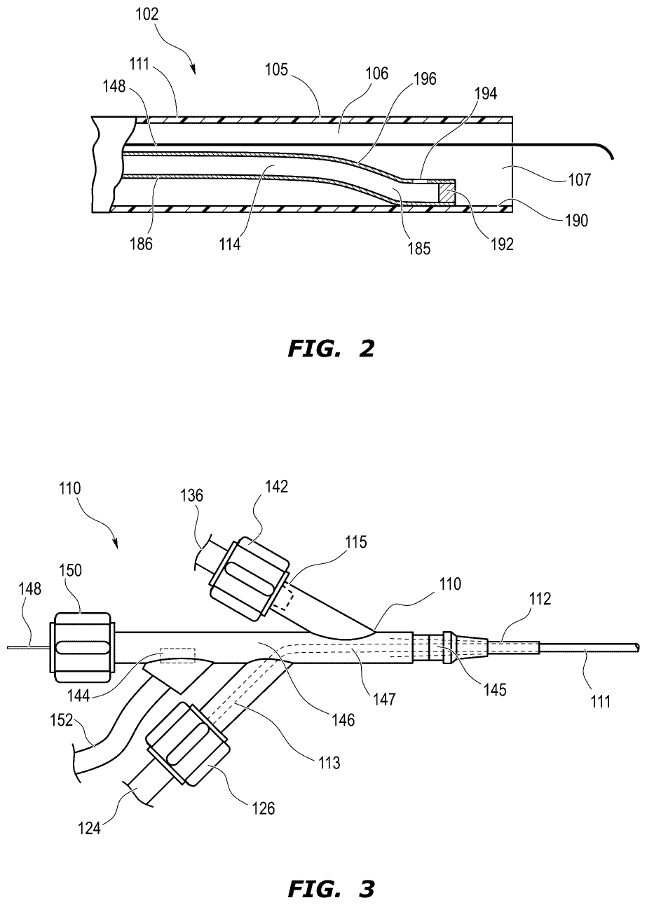

The aspiration catheter 102 has a distal end 105 and includes an over-the-wire guidewire lumen/aspiration lumen 106 extending between an open distal end 107, and a proximal end 108 comprising a y-connector 110. The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 includes a first female luer 113 which communicates with a catheter supply lumen 114 (FIG. 2), and a second female luer 115 which communicates with the guidewire lumen/aspiration lumen 106.

A spike 116 for coupling to a fluid source 20 (e.g., saline bag, saline bottle) allows fluid to enter through an extension tubing 118 and flow into a supply tube 119. An optional injection port 120 allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

Accessories 128 are illustrated that are intended for applying a vacuum source, such as a syringe 130 having a plunger 132 and a barrel 134, to the aspiration lumen 106 of the aspiration catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 3, a pressure transducer or sensor 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector 154. The connector 154 is plugged into a socket 156 of the pump 101. Pressure related signals may be processed by a circuit board 158 of the pump 101. The pressure transducer or sensor 144 may be powered from the pump 101, via the cable 152. The accessories 128 may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure transducer or sensor 144 thus senses a negative pressure and sends a signal, causing the controller to start the motor 182 of the pump 101. As the effect via the electronics is substantially immediate, the motor 182 starts pumping almost immediately after the pedal 166 is depressed. As the pedal 166 of the foot pedal 160 is released, the controller 174 then causes the pinch valve 162 to close. The pressure transducer or sensor 144 thus senses that no negative pressure is present and the controller 174 causes the motor 182 of the pump 101 to shut off. Again, the effect via the electronics is substantially immediate, and thus the motor 182 stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the aspiration catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure transducer or sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time, but may also be able to add durability to the vacuum line 136, for example, by not over-compressing. The foot pedal 160 may communicate with the pinch valve 162 via a wired connection through the pump 101 or may communicate with the pinch valve 162 wirelessly. Additionally, or alternatively, the pump may be controlled by buttons 184.

It should be noted that in certain embodiments, the pinch valve 162 and the foot pedal 160 may be incorporated for on/off operation of the pinch valve 162 on the vacuum line 136, without utilizing the pressure transducer or sensor 144. In fact, in some embodiments, the pressure transducer or sensor 144 may even be absent from the system 100 for aspirating thrombus, the foot pedal 160 being used as a predominant control means.

Turning to FIG. 2, a supply tube 186, which contains the catheter supply lumen 114, freely and coaxially extends within the over-the-wire guidewire lumen/aspiration lumen 106. At least a distal end 185 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 185 of the supply tube 186. The plug 192 blocks the exit of pressurized fluid, and thus the pressurized fluid is forced to exit through an opening or orifice 194 in the wall 196 of the supply tube 186. The free, coaxial relationship between the supply tube 186 and the catheter shaft 111 along their respective lengths (the supply tube 186 and the catheter shaft 111 being optionally coextensive, allows for improved flexibility. In some embodiments, in which a stiffer proximal end of the aspiration catheter 102 is desired (e.g., for pushability or even torqueability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

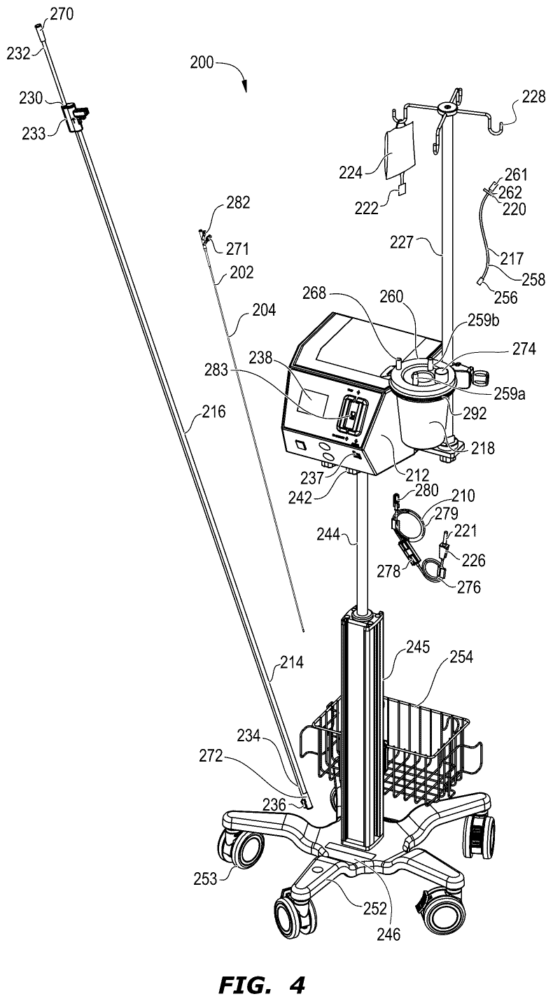

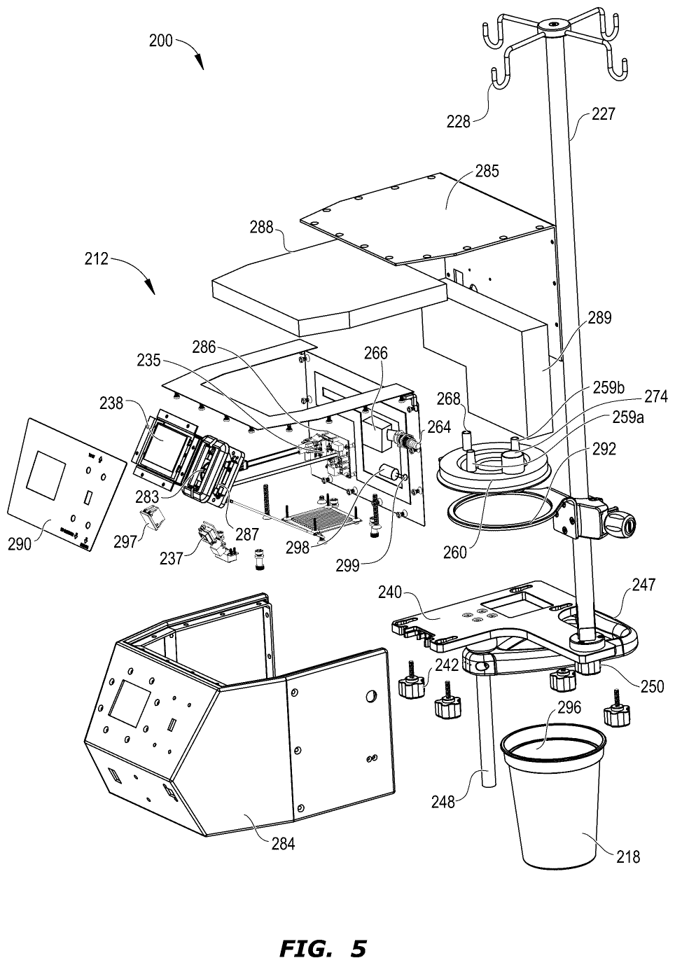

A system 200 for aspirating thrombus is illustrated in FIGS. 4-5. The system 200 for aspirating thrombus is similar to the system 100 and so the disclosure related to the system 100 is also applicable to the description of system 200. An aspiration catheter 202 is similar to the aspiration catheter 102 of FIGS. 1-3 and as such the description related to the aspiration catheter 102 of FIGS. 1-3 is also applicable to the description of the aspiration catheter 202.

The aspiration catheter 202 is configured for aspirating thrombus from peripheral vessels, but may also be configured with a size for treating coronary, cerebral, pulmonary or other arteries, or veins. The aspiration catheter 202/system 200 may be used in interventional procedures, but may also be used in surgical procedures. The aspiration catheter 202/system 200 may be used in vascular procedures, or non-vascular procedures (other body lumens, ducts, or cavities). The catheter 202 comprises an elongate shaft 204 configured for placement within a blood vessel of a subject; a catheter supply lumen 114 (FIG. 3) and a guidewire/aspiration lumen 106, each extending along the shaft, the supply lumen 114 having a proximal end 147 and a distal end 185, and the aspiration lumen 106 having a proximal end 145 (FIG. 3) and an open distal end 107 (FIG. 2); and an opening or orifice 194 at or near the distal end 185 of the supply lumen 114, the opening configured to allow the injection of pressurized fluid into the aspiration lumen 106 at or near the distal end 107 of the aspiration lumen 106 when the pressurized fluid is pumped through the supply lumen 114. In some embodiments, the opening or orifice 194 may be located proximal to the distal end 185 of the supply lumen 114. In some embodiments, the distal end 185 of the supply lumen 114 may comprise a plug 192. A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. The pressure sensor 230 is shown in FIG. 5 within a lumen at a junction between a first aspiration tube 232 and a control 233. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In alternative embodiments, the pressure sensor 230 may be replaced by another type of sensor that is configured to characterize fluid flow. In some embodiments, the sensor is a flow sensor, such as a Doppler flow velocity sensor.

The SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a second port 259 on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports 268. A user connects a first connector 270 of the sterile suction tubing 216 to an aspiration luer 271 of the aspiration catheter 202 (similar to luer 115), and connects the second connector 272 of the sterile suction tubing 216 to port 274 in the lid 260 of the canister 218. Connector 236 is then coupled to the mating receptacle 237 in the SDU 212 for communication with the control 233 and/or the pressure sensor 230. For instance, the connector 236 can be snapped into mating receptacle 237 in the SDU 212 for communication with elements of the control 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 121 is snapped in place, keeping the internal contents (e.g., saline) sterile. Systems configured for performing this type of sterile injection of high-pressure saline are described in U.S. Pat. No. 9,883,877, issued Feb. 6, 2018, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286. Noise from a motor 287 controlling the saddle 283 and from the vacuum pump 266 is abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 121 may couple to the saddle 283 by clipping, friction fitting, vacuum fitting, or other means.

FIG. 5 illustrates aspects pertaining to the vacuum canister 218, in which aspirant (e.g., thrombus, blood, saline) that is evacuated from the patient through the aspiration lumen 106 is collected. The canister 218 may be held in a canister mount 292 carried by the IV pole 227, or alternatively carried by any other part of the system 200. A lid 260 is configured to cover a portion of the canister 218, such as in a snapping manner, to close an interior 296 of the canister 218. Alternatively, the lid 260 may couple to the canister 218 by screwing, clipping, friction fitting, or other means.

The lid 260 may comprise two or more ports, including the first port 268 and second port 259 for providing negative pressure/vacuum to the aspiration lumen 106. For example, the lid 260 may comprise two ports, three ports, four ports, or more than four ports. Sterile suction tubing 216 may be connected to the lid 260 of the vacuum canister 218 at a first port 268 for transmitting a negative pressure to the sterile suction tubing 216 and to the aspiration lumen 106 of the aspiration catheter 102. Non-sterile suction tubing 217 may be connected to the lid 260 of the vacuum canister 218 at a second port 259 for providing a negative pressure to the vacuum canister 218. A negative pressure may be provided to the non-sterile suction tubing 217 (and to sterile suction tubing 216 and the aspiration lumen 106 connected therewith) by a vacuum source (e.g., a vacuum pump or syringe). The system 200 may also comprise means for sealing the two or more ports of the lid 260 when not in use, such as one or more port caps. A filter may be placed over an entry to the second port 259 so as to prevent aspirant from traveling along the non-sterile suction tubing 217 from the vacuum canister 218 to the vacuum source.

The vacuum canister 218 preferably has a sufficient volumetric capacity for receiving all aspirant collected during the surgical procedure. Receptacles having a volumetric capacity of approximately 100 cubic inches, or receptacles having a diameter of approximately 5.0 inches and a height of approximately 7.0 inches, have been found to provide sufficient volumetric capacity.

Returning to FIG. 4, a solenoid 298 is carried internally in the SDU 212, and is configured to interface with the interior 296 of the canister 218, via the suction tubing 214, or via any additional tubing. The solenoid 298 is configured to vent the negative pressure inside the canister 218, by opening a valve 299 coupled to the solenoid (mechanically or electromagnetically) that opens the interior 296 of the canister 218 to ambient pressure. The venting allows any foaming of blood or fluid, such as any aspirated liquid, within the canister 218 to be reduced. Foaming can occur during a thrombolysis procedure due to cavitation, as air bubbles are formed. The solenoid 298 is then configured to close the valve 299, to allow negative pressure to again be built up within the interior 296 of the canister 218. The controller 235 is configured to automatically energize the solenoid 298, in order to allow for the degassing/defoaming. For example, the controller 235 may send a signal to energize the solenoid 298 based on the measurement of a targeted negative pressure and/or a targeted time of aspiration cycle. In other cases, the controller 235 can send a signal to energize the solenoid 298 every minute, every five minutes, every ten minutes, etc. Additionally, a user can operate the controller 235, and more generally the controller 174, of the system 200 through the interface panel 290 to initiate degassing/defoaming of the interior 296. The venting may also be able to remove air bubbles inside the other lumens of the catheter and tubing sets.

In some embodiments, the controller 235 can output or send a signal to energize the solenoid 298 to open the valve 299, in order to stop any aspiration, while still allowing the SDU 212 to deliver saline, medication, or saline combined with medication (e.g., thrombolytic drugs), so that the fluids can be delivered out of the open distal end 107 (instead of being aspirated through the aspiration lumen 106).

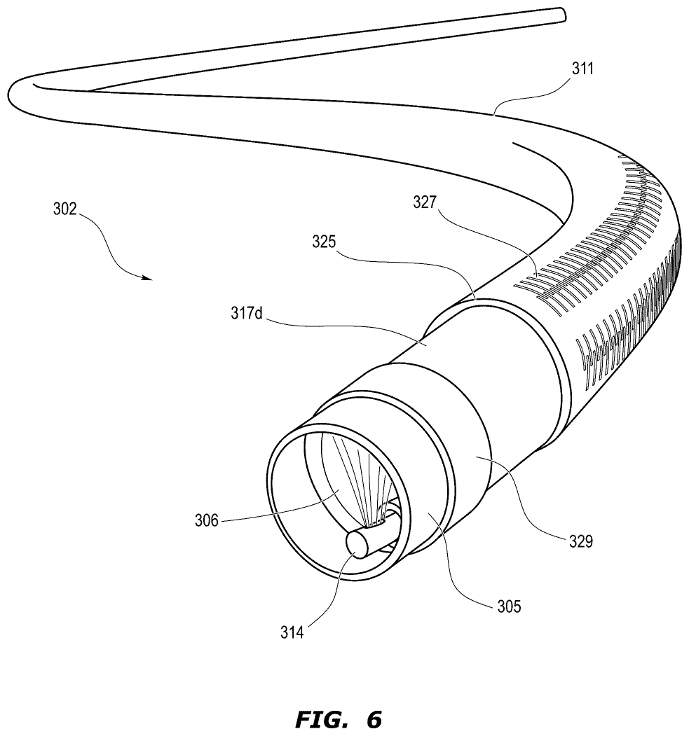

Turning to FIGS. 6-8 illustrated is another configuration of an aspiration catheter 302 that can be used with the systems 100 or 200 for aspirating thrombus. As such, the discussions related to the aspiration catheter 102 and the aspiration catheter 202 are also applicable to the aspiration catheter 302 illustrated in FIGS. 6-8.

As shown in more detail in FIGS. 6-8, aspiration catheter 302 includes an aspiration lumen 306 formed by a shaft 311, such as a hypotube, jacketed by a polymer jacket or a polymer jacket is laminated on the hypotube. For instance, a shaft body 317a is illustrated being jacketed by a jacket 317b, such as by heat-shrinking, laminating or otherwise applying the jacket 317b to the shaft body 317a. A distal end 305 of the aspiration catheter 302 includes a multilayer structure. A portion 317c of the jacket 317b extends distally of a shaft distal end 325 of the shaft 311 to form part of the distal end 305. An outer layer or outer jacket 317d overlaps the jacket 317b, extends towards and overlaps a shaft distal end 325, and forms the aspiration catheter distal end 305a or the distal most end of the aspiration catheter with the distal opening 307. The outer jacket 317d protects a distal portion 385 of a supply tube 386 containing a supply lumen 314. While reference is made of a multilayer structure, it will be understood that one or more layers can be omitted or combined together. Additionally, one or more of the layers or shaft body can include braided or other members to increase strength and/or flexibility. Alternatively, the shaft and associated layers can be formed by extruding the shaft or using other structures to form the shaft. For instance, the shaft 311 can be a single or multi-lumen extruded polymer shaft.

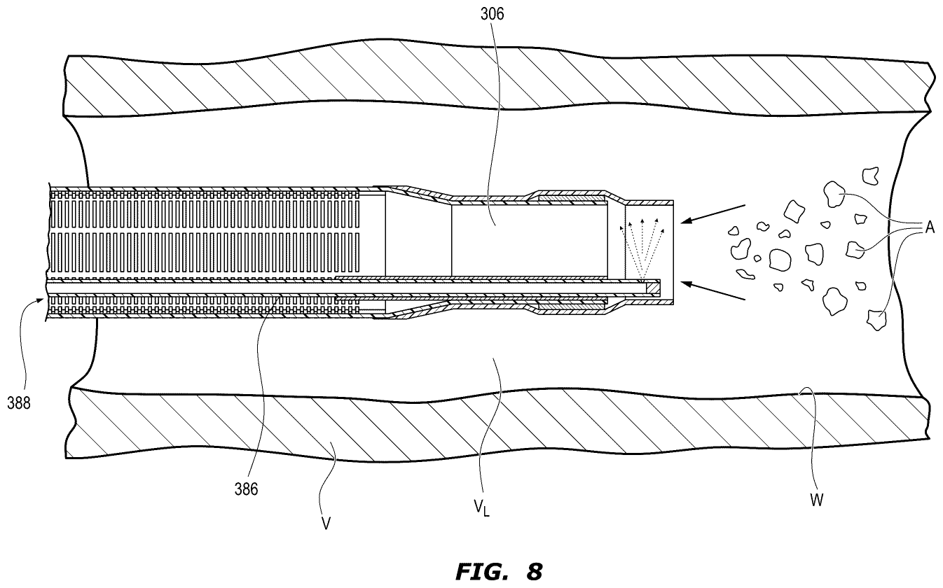

The supply lumen 314 may be configured to provide a high pressure fluid injection, such as saline, within the aspiration lumen 306 (optionally coextensive with the supply lumen 314) for macerating a thrombus as it is aspirated, such as illustrated in FIG. 8 where the aspiration catheter 302 is disposed within a vessel lumen VL of a vessel V and aspirant A is being drawing into the aspiration lumen 306. The saline injection may occur through orifice 394 near the distal end of the supply lumen 314 if the opening of the supply lumen is plugged with a plug similar to plug 192 (FIG. 2). Aspiration catheter 302 may also include a radiopaque (RO) ring 329 at or near the distal end 305 of aspiration catheter 302 for identifying the location of aspiration. The radiopaque ring 329 optionally encircles the aspiration catheter 302. In the illustrated configuration, the RO ring 329 is disposed between the jacket 317b and outer jacket 317d. The RO ring 329 can be formed of any suitable radiopaque material, such as tantalum, tungsten, platinum/iridium, gold, silver, and combinations or modifications thereof.

The shaft 311 can include one or more openings 327 to increase a flexibility of shaft 311 to aid with advancement of the aspiration catheter 302 through the tortuous anatomy of a patient. While reference is made to a “hypotube,” it will be understood that other tubular structures can be used for the shaft 311. Additionally, the shaft 311 can be formed from polymers, metals, alloys, braided structures, coiled structures, and combinations or modifications thereof. The shaft 311 can alternatively be a single or multi-lumen extruded polymer shaft. Furthermore, the jacket 317b and outer jacket 317d can be formed of a variety of polymers and copolymers, plastics, PEBAX, HYTREL, rubber, nylon, polyethylene, polyurethane, polyester, polymer shrink tubing, such as polytetrafluoroethylene, fluorinated ethylene propylene, etc. and combinations or modifications thereof.

As illustrated in FIG. 8, the supply tube 386 is fixed within the distal end 305 of the shaft 311, with a proximally extending portion 388 of the supply tube 386 extending in a free-floating manner proximally along the shaft 311. When thrombus is drawn through the distal opening 307 into the aspiration lumen 306 of the aspiration catheter 302 and the pressurized fluid or jet of fluid partially macerates the thrombus, a number of larger pieces of thrombus might be allowed to advance along the aspiration lumen 306. As the supply tube 386 moves within the aspiration lumen 306 under the influence of the aspirant flow, thrombus might contact the supply tube 386, become entangled with the supply tube 386, and potentially block the aspiration lumen 306 of the aspiration catheter 302. Positioning the supply tube 386 to one side of the aspiration lumen 306 or within a separate supply tube lumen reduces the possibility of inadvertent aspiration lumen blockage through entanglement between the supply tube and aspirants, including larger pieces of thrombus.

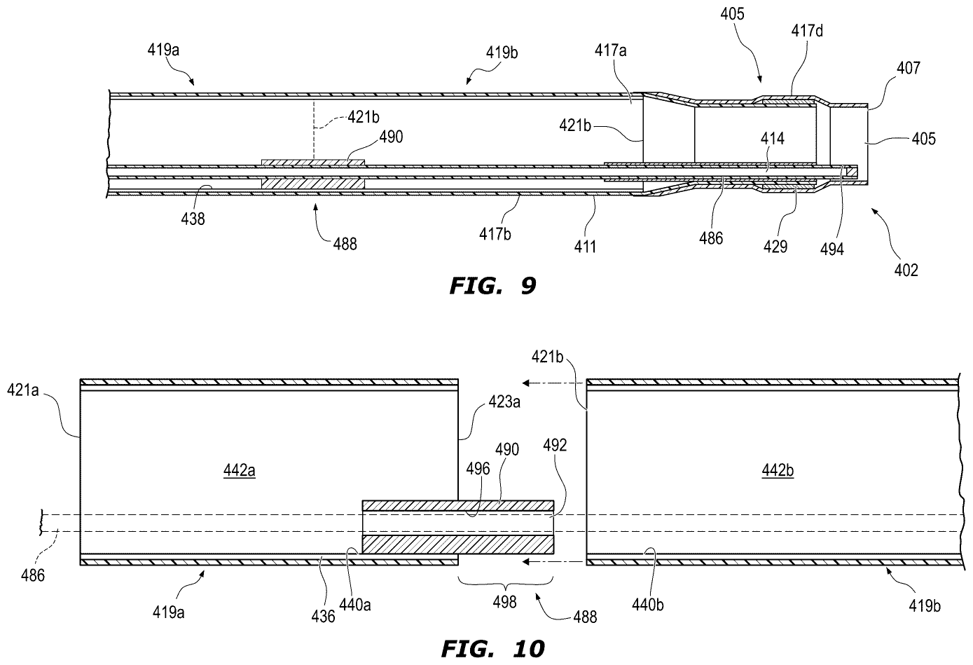

Turning to FIGS. 9-10 illustrated is another aspiration catheter 402 that limits the possibility of inadvertent aspiration lumen blockage through entanglement between the supply tube and aspirants, including larger pieces of thrombus. The aspiration catheter 402 can be used with the systems 100 or 200 for aspirating thrombus. As such, the discussion relates to the aspiration catheter 102, 202, and 302 are also applicable and related to the aspiration catheter 402. Like reference numerals are used for like structure and/or elements with like functions. For simplicity of description, only a portion of the aspiration catheter 402 is depicted in FIGS. 9-10 with it understood that other portions of the aspiration catheter 402 can have the same or similar structures and functions to the any of the aspiration catheters 102, 202, and 302. For instance, the proximal ends, distal ends, and any other structures, etc. of the aspiration catheter, shaft, and supply tube, with associated jackets, etc. of any of the aspiration catheters 102, 202, and can be included in aspiration catheter 402.

As illustrated, aspiration catheter 402 includes an aspiration lumen 406 formed by a shaft 411, such as a hypotube, jacketed by a polymer jacket or a polymer jacket is laminated on the hypotube. For instance, a shaft body 417a is illustrated being jacketed by a jacket 417b, such as by heat-shrinking, laminating or otherwise applying the jacket 417b to the shaft body 417a. A supply tube 486 having the supply lumen 414 extends through the aspiration lumen 406 of the shaft body 417a towards the distal opening 407. The supply lumen 414 may be configured to provide a high pressure fluid injection, such as saline, within the aspiration lumen 406 for macerating a thrombus as it is aspirated. The saline injection may occur through orifice 494 near the distal end of the supply lumen 414 if the opening of the supply lumen is plugged with a plug similar to plug 192 (FIG. 2). Aspiration catheter 402 may also include a radiopaque ring 429 at or near the distal end 405 of aspiration catheter 402 for identifying the location of aspiration. The radiopaque ring 429 optionally encircles the aspiration catheter 402. The radiopaque ring 429 can be formed of any suitable radiopaque material, such as tantalum, tungsten, platinum/iridium, gold, silver, and combinations or modifications thereof.

The shaft 411 can include one or more slits or a spiral cut (e.g., an interrupted spiral cut) to increase a flexibility of shaft 411 to aid with advancement of the aspiration catheter 402 through the tortuous anatomy of a patient. While reference is made to a “hypotube,” it will be understood that other tubular structures can be used for the shaft 411. Additionally, the shaft 411 can be formed from polymers, metals, alloys, braided structures, coiled structures, flat coiled structures, and combinations or modifications thereof. The shaft 411 can alternatively be a single or multi-lumen extruded polymer shaft. Furthermore, the jacket 417b and outer jacket 417d can be formed of a variety of polymers and copolymers, plastics, PEBAX, HYTREL, rubber, nylon, polyethylene, polyurethane, polyester polymer shrink tubing, such as polytetrafluoroethylene, fluorinated ethylene propylene, etc. and combinations or modifications thereof.

As illustrated in FIG. 9, the supply tube 486 is mounted or disposed towards one side of the aspiration lumen 406, thereby limiting the impact of the supply tube 486 upon flow of aspirant/thrombus along the aspiration lumen 406. In the illustrated configuration, disposed within the aspiration lumen 406 are supply tube mounts 488 that are fixed to a wall 436 of the shaft body 417a or to an inner surface 438 of the aspiration lumen 406. Each supply tube mount 488 includes a mount body 490 with a mount orifice 492 extending along a length of the mount body 490. An inner surface 496 of the mount orifice 492 can be coated with a lubricous material to aid with movement of the supply tube 486 within the supply tube mounts 488 as the aspiration catheter 402 is advanced and moved within the tortuous anatomy of the patient. For instance, as the aspiration catheter 402 is turned or bends, the supply tube 486 can slide within the mount orifice 492. The lubricious material or coating can include silicone coatings, polymeric coatings, hydrophilic coatings, polytetrafluoroethylene (PTFE), combinations or modifications thereof. Alternatively, the inner surface 496 of the mount orifice 492 can include a uniform or non-uniform structures that reduce frictional engagement between the supply tube 486 and the inner surface 496.

The supply tube mounts 488 can be disposed within the aspiration catheter 402 at various locations along a length of the aspiration catheter 402 or the shaft body 417a. For instance, a distance between supply tube mounts 488, such as adjacent or closely positioned supply tube mounts 488 along the length of the aspiration catheter 402, can range from about 1 inch to about 30 inches, from about 5 inches to about 25 inches, from about 10 inches to about 20 inches, from about 15 inches to about 20 inches, or is in a range between any two of the foregoing. The distance between two adjacent supply tube mounts 488 can be uniform or non-uniform along the length of the aspiration catheter 402 or the shaft body 417a. For instance, a spacing between two adjacent supply tube mounts 488 can increase in a proximal-to-distal direction along the aspiration catheter 402 or the shaft body 417a to allow more movement of the supply tube 486 with the aspiration lumen 406. Alternatively, a spacing between two adjacent supply tube mounts 488 can decrease in a proximal-to-distal direction along the aspiration catheter 402 or the shaft body 417a to decrease movement of the supply tube 486 within the aspiration lumen 406. To avoid any projecting edge or ledge between the mount body and the supply tube 486 it is possible to “bridge” the distance between adjacent mount bodies 490 with a coil. Such a coil can be a flat wire coil or the wire forming the coil could have any other cross section. In a case where the mount body is made from metal the coil can be laser welded to the mount body. Such a flat wire coil “bridge” is shown and described in conjunction with FIG. 10B. To reduce friction of the lumen running through the coil, the wire forming the coil can be coated before coiling (e.g., with a lubricious coating).

As illustrated, the aspiration catheter 402 is formed as a hypotube. To aid with positioning the supply tube mounts 488 within the aspiration lumen 406 of the aspiration catheter 402, the shaft body 417a can be formed of a plurality of shaft body segments 419 that can be coupled of joined, coupled, or attached together, such as via welding, laser welding, soldering, thermal bonding, chemical bonding, glues, adhesives, mechanical fasteners, or combinations or modifications, to form the shaft body 417a. Each shaft body segment 419 can include a first end 421, a second end 423, and a shaft body segment inner surface 440 extending from the first end 421 to the second end 423. The shaft body segment inner surface 440 with those inner surface segments of the shaft body segment other shaft body segments form the inner surface 438 of the shaft body 417a. The shaft body segment inner surface 440 also forms an outer periphery of a shaft body segment lumen (e.g., labeled 442a, 442b in FIG. 10). As with the inner surface segments, the segment lumens 442a, 442b with those segment lumens of the other shaft body segments form the aspiration lumen 406 of the shaft body 417a.

The supply tube mount 488 not only can be used to guide the supply tube 486, the supply tube mount 488 can also be used to align adjacent shaft body segments 419 during manufacture or processing of the shaft body 417a. For instance, and as illustrated in FIGS. 9 and 10, a supply tube mount 488 can joined, coupled, or attached to a shaft body segment inner surface 440a of the shaft body segment 419a. With the supply tube mount 488 joined, coupled, or attached to the shaft body segment 419a, the supply tube mount 488 extends from the second end 423a in a cantilever fashion or manner, i.e., includes a cantilevered portion 498. The supply tube mount 488 can be received within a first end 421b of a shaft body segment 419b and by placing the supply tube mount 488 in contact with an shaft body segment inner surface 440b, the second end 423a of the shaft body segment 419a is aligned with the first end 421b of the shaft body segment 419b for joining, coupling, or attaching the shaft body segment 419a to the shaft body segment 419b. Therefore, the supply tube mount 488 aligns the second end 423a of the shaft body segment 419a is aligned with the first end 421b of the shaft body segment 419b for welding, for instance. By repeating this process for other shaft body segments, any length of aspiration catheter 402 and shaft body 417a can be formed.

When the second end 423a and the first end 421b are joined, coupled, or attached, such as by welding for example, the cantilevered portion 498 of the supply tube mount 488 can optionally remain detached from the shaft body segment inner surface 440b of the shaft body segment 419b, i.e., not fixed to the shaft body segment inner surface 440b but moveable in relation to the shaft body segment inner surface 440b. For instance, the supply tube mount 488 is attached to the shaft body segment 419a or the shaft body segment 419b but not both the shaft body segment 419a or the shaft body segment 419b. Maintaining movement of the cantilevered portion 498 in relation to the shaft body segment inner surface 440b and the shaft body segment 419b, flexibility of the shaft body 417a is maintained. In other configurations, the cantilevered portion 498 is attached, coupled, or joined to the shaft body segment inner surface 440b as the second end 423a of the shaft body segment 419a is attached, coupled, or joined to the first end 421b of the shaft body segment 419b.

Optionally, a mandrel or other structure (illustrated in phantom) can be disposed within the shaft body segment lumen 442a of the shaft body segment 419a and a shaft body segment lumen 442b of the shaft body segment 419b to align the shaft body segment 419a and the shaft body segment 419b so that the second end 423a of the shaft body segment 419a can be attached, coupled or joined to the first end 421b of the shaft body segment 419b. As mentioned above, by repeating this process for other shaft body segments, any length of aspiration catheter 402 and shaft body 417a can be formed.

Each individual shaft body segment 419 can have a length from 1 inch to about 30 inches, from about 5 inches to about 25 inches, from about 10 inches to about 20 inches, from about 15 inches to about 20 inches, or is in a range between any two of the foregoing. The lengths of those shaft body segments used to form the shaft body 417a can be uniform or non-uniform along a length of the shaft body 417a. For instance, a length of two adjacent shaft body segments can increase in a proximal-to-distal direction along the aspiration catheter 402 or the shaft body 417a. Alternatively, a length of two adjacent shaft body segments can decrease in a proximal-to-distal direction along the aspiration catheter 402 or the shaft body 417a.

As an alternative to use of a mount body to keep a supply tube inside of a laser cut hypotube, pushed to one side, yet another embodiment may laser weld a flexible metal member to the inside of the hypotube. The flexible inner member can comprise a coil structure or a cable tube. A coil structure can use wires of various shapes such as round wires, flat wires, or other shapes. Of such options, a flat wire may provide the smoothest surface and therefore have the least interaction with an aspirant. To create an even smoother surface the flat wire could be centerless ground before attachment to the wall of the catheter. As it is very difficult to laser weld from the hypotube interior, the coil can be tagged to the side of the hypotube from the outside diameter. It is important that the coil be in close contact with the interior wall of the laser cut hypotube. This can be achieved in a variety of ways. For example, it is possible to insert and inflate a balloon on the inside diameter of said laser cut hypotube, pressing the coil against the wall, ensuring that there will be substantially no gap when laser welding. Another option would be to insert a spring-loaded element which is pulled through the inside diameter, pressing the coil against the interior wall.

It is possible to use laser welding to weld through both the outer surface of the laser cut hypotube and the coil pressed against the interior wall. However alignment can be difficult. By way of example, another way to attach the coil to the interior wall of a laser cut hypotube is to form small holes (e.g., during laser cutting) through the hypotube, with a diameter of less than 1 mm, e.g., from 0.1 mm to 0.5 mm). Such holes can be used to align the hypotube with the interior coil using magnification (e.g., a microscope or strong magnifying glass). A welding laser may then weld the inner coil to the wall of the hypotube. While a flat wire coil may be preferred, different shape wire may alternatively be used. To decrease friction the wire may be coated before coiling e.g., using a PTFE powder coating. To make sure that the catheter is still flexible the coil should be stretched somewhat to allow for compression. The amount of stretching should be limited to a minimum to minimize or avoid gaps between the coil elements which might interfere with the aspirant flowing through the aspiration lumen. Beside coils which can be welded to the inside diameter of the hypotube it is also possible to laser weld cable tubes to the inside diameter. Such tubes are very flexible and depending on their construction will stretch as well. To minimize wall thickness and allow for stretching, a dual layer cable tube (e.g., HSS tube) may be preferred.

FIGS. 10A-10B illustrate an example of how a flat wire coil may be welded into the inside of a hypotube 417a. For example, FIG. 10A shows a flat view of such a hypotube, showing the formation of rows of small holes 462 formed through such hypotube (e.g., during laser cutting). Such holes 462 are shown enlarged and not to scale, for illustrative purposes. By way of example, a plurality of holes may be formed in the hypotube circumference, over any desired length within which the wire coil is to be secured within the hypotube. By way of example, the number of holes included in a given circumferential row may be from 2 to 10, such as 3 to 8, or about 4 to 6. Spacing between adjacent rows may vary, e.g., ranging from 5 mm to 50 mm, such as from 10 mm to 30 mm (e.g., about 20 mm). FIG. 10B illustrates a cross-sectional view into such a hypotube 417a, showing the flat wire coil 473, which may be attached to the interior surface of the hypotube 417a through holes 462 (e.g., through laser welding through such holes). In an embodiment, the supply tube mount may simply comprise such a flat wire coil. In another embodiment, such a flat wire coil may “bridge” the space between adjacent supply tube mounts 488, reducing or eliminating any ledge that may otherwise be associated with the tube mounts, as described in conjunction with FIG. 10. A similar plug or slot weld configuration is shown and described in conjunction with FIGS. 16-18. While the flat wire coil 473 may be used to house a supply tube with a supply lumen for delivering pressurized fluid, it could also or alternative be used to house any other desired structure (e.g., guidewire, or other elongate structures).

The aspiration catheter 402, therefore, includes supply tube mounts or other structures that can be used to position a supply tube of an aspiration system to limit or prevent blockage of an aspiration lumen of an aspiration catheter during aspiration of thrombus, clots, or other aspirants. Positioning the supply tube mount in aspiration catheter 402 maintains the supply tube in a desired location against the wall of the aspiration catheter and so limits or prevents blockage of the aspiration lumen of the aspiration catheter 402 during aspiration of thrombus, clots, or other aspirants.

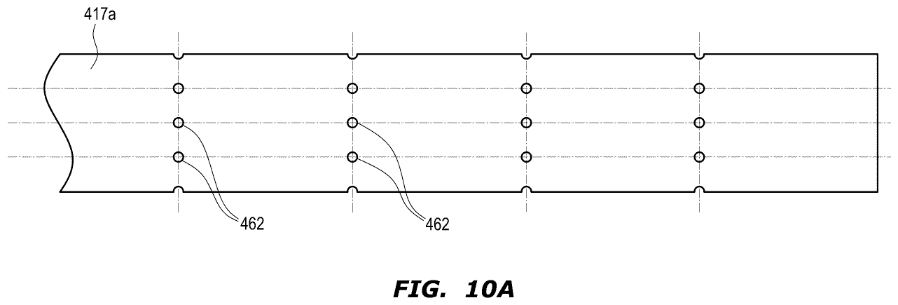

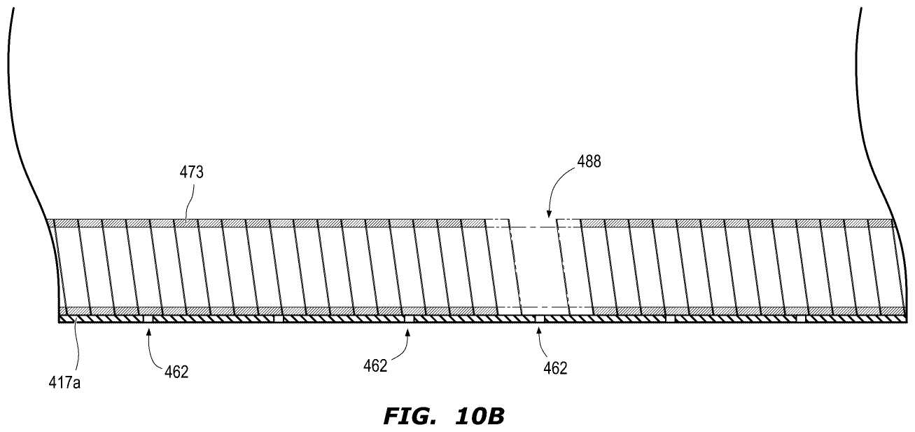

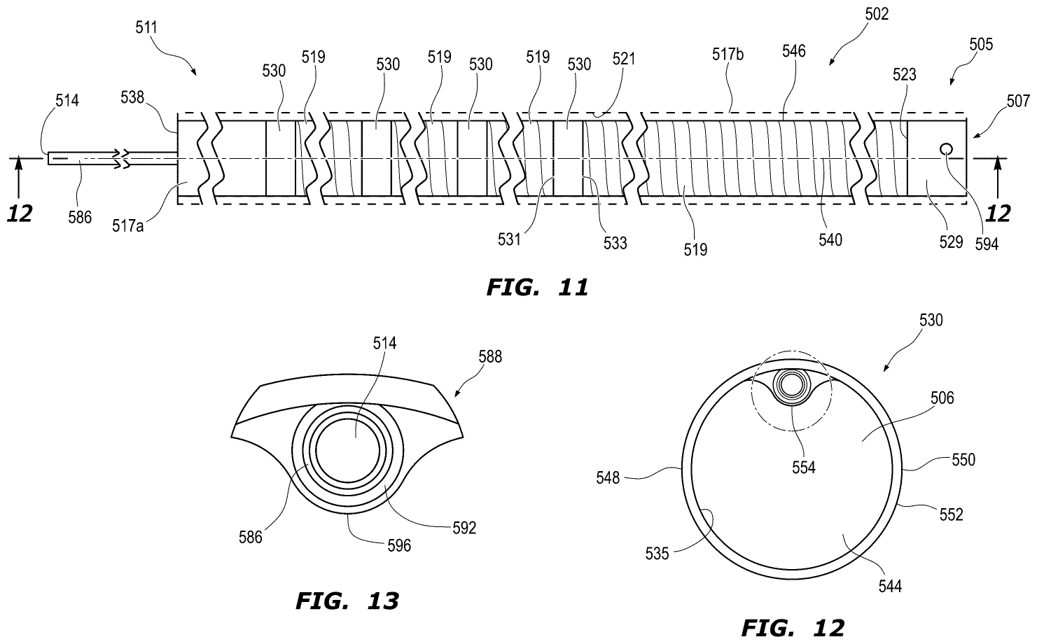

Turning to FIGS. 11-14 illustrated is another an aspiration catheter 502 that limits the possibility of inadvertent aspiration lumen blockage through entanglement between the supply tube and aspirants, including larger pieces of thrombus. The aspiration catheter 502 can be used with the systems 100 or 200 for aspirating thrombus. As such, the discussion relates to the aspiration catheter 102, 202, 302, and 402 are also applicable and related to the aspiration catheter 502. Like reference numerals are used for like structure and/or elements with like functions. For simplicity of description, only a portion of the aspiration catheter 502 is depicted in FIGS. 11-14 with it understood that other portions of the aspiration catheter 502 can have the same or similar structures and functions to the any of the aspiration catheters 102, 202, 302, or 402. For instance, the proximal ends, distal ends, and any other structures, etc. of the aspiration catheter, shaft, and supply tube, with associated jackets, etc. of any of the aspiration catheters 102, 202, 302, or can be included in aspiration catheter 502.

As illustrated in FIGS. 11-14, aspiration catheter 502 includes an aspiration lumen 506 formed by a shaft 511, such as a coiled structure, jacketed by a polymer jacket or a polymer jacket. For instance, a shaft body 517a is illustrated being jacketed by a jacket 517b, such as by heat-shrinking, laminating or otherwise applying the jacket 517b to the shaft body 517a. A supply tube 586 having the supply lumen 514 extends through the aspiration lumen 506 of the shaft body 517a towards the distal opening 507. The supply lumen 514 may be configured to provide a high pressure fluid injection or jet, such as saline, within the aspiration lumen 506 for macerating a thrombus as it is aspirated. The saline injection may occur through an orifice near the distal end of the supply lumen 514 if the opening of the supply lumen is plugged with a plug similar to plug (similar to plug 192 of FIG. 2). Aspiration catheter 502 may also include a radiopaque ring 529 at or near the distal end 505 of aspiration catheter 502 for identifying the location of aspiration. The radiopaque ring 529 optionally encircles the aspiration catheter 502. The radiopaque ring 529 can be formed of any suitable radiopaque material, such as tantalum, tungsten, platinum/iridium, gold, silver, and combinations or modifications thereof.

While reference is made to the shaft 511 being a coiled structure, it will be understood that other tubular structures can be used for the shaft 511. For instance, the shaft 511 can be a hypotube as described in relation to shafts 311 and 411. Additionally, the shaft 511 can be formed from polymers, metals, alloys, braided structures, flat coiled structures, and combinations or modifications thereof. The shaft 511 can alternatively be a single or multi-lumen extruded polymer shaft. Furthermore, the jacket 517b and any outer jacket can be formed of a variety of polymers and copolymers, plastics, PEBAX, HYTREL, rubber, nylon, polyethylene, polyurethane, polyester, polymer shrink tubing, such as polytetrafluoroethylene, fluorinated ethylene propylene, etc. and combinations or modifications thereof.

As illustrated in FIGS. 11-14, the supply tube 586 is mounted or disposed towards one side of the aspiration lumen 506, thereby limiting the impact of the supply tube 586 upon flow of aspirant/thrombus along the aspiration lumen 506. The aspiration catheter 502, or the shaft body 517a, is formed of a plurality of shaft body segments 519 and a plurality of supply tube mounts, such as supply tube mount segments 530 that are joined, coupled, or attached together, such as via welding, laser welding, soldering, thermal bonding, chemical bonding, glues, adhesives, mechanical fasteners, or combinations or modifications, to form the shaft body 517a. For instance, each shaft body segment 519 can join, couple or attached to one or two other shaft body segments 519 and/or one or two other supply tube mount segments 530 to form the shaft body 517a. Similarly, each supply tube mount segment 530 can join, couple or attached to one or two other supply tube mount segments 530 and/or one or two other shaft body segments 519 to form the shaft body 517a. By repeating this process for joining, coupling, or attaching the shaft body segments and supply tube mount segments, any length of aspiration catheter 502 and shaft body 517a can be formed. Additionally, forming the aspiration catheter 502 by joining, coupling, or attaching the shaft body segments and supply tube mount segments can also include positioning through or slidably engaging the supply tube 586 with mount orifices 592 associated with the supply tube mount segments 530. Advancing or positioning the supply tube 586 within the mount orifices 592 can occur during or after formation of the shaft body 517a.

As illustrated in FIG. 11, the shaft body segment 519 includes a first shaft body segment end 521, a second shaft body segment end 523, and a shaft body segment inner surface 540 extending from the first shaft body segment end 521 to the shaft body segment second end 523. The supply tube mount segment 530 can be joined, coupled or attached to the shaft body segment 519. The supply tube mount segment 530 can include a first supply tube mount segment end 531, a second supply tube mount segment end 533, and a supply tube mount segment inner surface 535 extending from the first supply tube mount segment end 531 to the second supply tube mount segment end 533. A shaft body segment inner surface 540 with those shaft body segment inner surface of the other shaft body segments, and a supply tube mount segment inner surface 540 with those supply tube mount segment inner surfaces of other shaft tube mount segments form the inner surface 538 of the shaft body 517a. The inner surface 538 also forms an outer periphery of the aspiration lumen 506. As with the inner surface segments, a shaft body segment lumen 542 with those shaft body segment lumens of the other shaft body segments, and a supply tube mount segment lumen 544 with those supply tube mount segment lumens of other shaft tube mount segments form the form the aspiration lumen 506 of the shaft body 517a.

To provide general uniformity to the aspiration lumen 506 and the aspiration catheter 502, a supply tube mount segment inner surface 535 approximates the shaft body inner surface 538 to provide and form the aspiration lumen 506. A shaft body segment outer surface 546 approximates a supply tube mount segment outer surface 548 to provide a smooth transition between the shaft body segment 519 and the supply tube mount segment 530. Stated another way, the supply tube mount segment 530 has an outside diameter that substantially matches an outside diameter of the shaft body segment 419, the inside diameter of the supply tube mount segment 530 substantially matches the inside diameter of the shaft body segment 519, at least a portion of the supply tube mount segment lumen 544 approximates or matches at least a portion of the shaft body segment lumen 542, and at least a portion of the shaft body segment outer surface 546 approximates or matches at least a portion of the supply tube mount segment outer surface 548.

To vary a flexibility and positioning capabilities of the aspiration catheter 502, lengths of the shaft body segments 519 and optionally the supply tube mount segments 530 can be varied along a length of the aspiration catheter 502. For instance, the lengths of those shaft body segments used to form the shaft body 517a can be uniform or non-uniform along a length of the shaft body 517a. For instance, lengths of two adjacent shaft body segments can increase in a proximal-to-distal direction along the aspiration catheter 502 or the shaft body 517a. Alternatively, a length of two adjacent shaft body segments can decrease in a proximal-to-distal direction along the aspiration catheter 502 or the shaft body 517a. Similarly, lengths of two adjacent supply tube mount segments can increase in a proximal-to-distal direction along the aspiration catheter 502 or the shaft body 517a. Alternatively, a length of two adjacent supply tube mount segments 530 can decrease in a proximal-to-distal direction along the aspiration catheter 502 or the shaft body 517a.

Each individual shaft body segment 519 can have a length from about 1 inch to about 30 inches, from about 5 inches to about 25 inches, from about 10 inches to about 20 inches, from about 15 inches to about 20 inches, or is in a range between any two of the foregoing. Each individual supply tube mount segment 530 can have a length from about 0.025 inches to about 0.5 inches, from about 0.03 inches to about 0.25 inches, from about 0.04 inches to about 0.25 inches, from about 0.05 inches to about 0.25 inches, about 0.1 inches, or is in a range between any two of the foregoing.

With reference to FIGS. 11-14, the supply tube mount segment 530 is disposed between two shaft body segments 519. The supply tube mount segment 530 can have a disk-like structure. The supply tube mount segment 530 includes a supply tube mount body 550, the supply tube mount segment lumen 544, and the mount orifice 592. A mount wall 552 of the supply tube mount body 550 includes a protruding portion 554 that extends or protrudes inwardly to accommodate the mount orifice 592 that receives the supply tube 586. However, in other configurations the mount orifice 592 can be formed in the mount wall 552 without including the protruding portion 554 and so the supply tube mount segment lumen 544 has a generally uniform diameter or shape.

By disposing a plurality of the supply tube mount segments 530 along a length of the shaft body 517a, the mount orifices 592 retain the supply tube 586 towards a side of the aspiration lumen 506 as the aspiration catheter 502 is advanced and moved within the tortuous anatomy of the patient. To aid with movement of the supply tube 586 within the mount orifices 592, an inner surface 596 of the mount orifice 592 can be coated with a lubricous material. As the aspiration catheter 502 is turned or bends, the supply tube 586 can slide within the mount orifice 592. The lubricious material or coating can include silicone coatings, polymeric coatings, hydrophilic coatings, polytetrafluoroethylene (PTFE), combinations or modifications thereof. Alternatively, the inner surface 596 of the mount orifice 592 can include a uniform or non-uniform structure that reduces frictional engagement between the supply tube 586 and the inner surface 596.

A plurality of supply tube mount segments 530 can be included in the aspiration catheter 502 at various locations along a length of the aspiration catheter 502 or the shaft body 517a. These supply tube mount segments 530 are joined, coupled, or attached between shaft body segments 519, in a similar manner as described with respect to aspiration catheter 402. For instance, a second supply body segment end 523a of shaft body segment 519a can be joined, coupled, or attached to the first supply tube mount segment end 531 of the supply tube mount segment 530. The supply tube mount segment 530 is then in turn joined, coupled, or attached to the first shaft body segment end 521b of a shaft body segment 519b. This process is repeated until a desired length for the aspiration catheter. As the shaft body segments and supply tube mount segments are joined, a tether 560 is passed through the mount orifices 592 to provide access to the mount orifices 592 for positioning the supply tube 586. For instance, once a desired length of the aspiration catheter is achieved, a tether end of the tether 560 is temporarily attached to the supply tube 586. As the tether 560 is proximally withdrawn from the shaft body 517a, the supply tube 586 is drawn through the mount orifices 592 along the length of the aspiration catheter. Once, for instance, the orifice 594 is positioned at the distal end 505 of the shaft body 517a, it can be fixed within the aspiration lumen 506. The tether 560 can be moved proximally and/or distally to position the orifice 594. In still another configuration, the process of joining, coupling or attaching one or more shaft body segments and supply tube mount segments is repeated until a length of the aspiration catheter less than a full length of the aspiration catheter is achieved and the supply tube 586 is advanced through one or more mount orifices 592 of the supply tube mount segments without the aid of the tether 560. It will also be understood that any other combination of using the tether 560 and forming portions of the aspiration catheter are possible.

Optionally, a mandrel or other structure can be used during joining, coupling, or attaching the shaft body segment 519 to the supply tube mount segment 530 or the shaft body segment 519 to another shaft body segment 519. For instance, a mandrel or other structure can be disposed within the shaft body segment lumens of the shaft body segment 519 and a supply tube mount segment lumen 544 of the supply tube mount segment 530 to align the shaft body segment 519 and the supply tube mount segment 530 so that they can be attached, coupled or joined together.

A distance between two adjacent supply tube mount segments 530 can range from about 1 inch to about 30 inches, from about 5 inches to about 25 inches, from about 10 inches to about 20 inches, from about 15 inches to about 20 inches, or is in a range between any two of the foregoing. The distance between two adjacent supply tube mount segments 530 can be uniform or non-uniform along the length of the aspiration catheter 502 or the shaft body 517a. For instance, a spacing between two supply tube mounts 588, adjacent along a length of the aspiration catheter 502, can increase in a proximal-to-distal direction along the aspiration catheter 502 or the shaft body 517a to allow more movement of the supply tube 586 with the aspiration lumen 506. Alternatively, a spacing between two adjacent supply tube mount segments 530 can decrease in a proximal-to-distal direction along the aspiration catheter 502 or the shaft body 517a to decrease movement of the supply tube 586 within the aspiration lumen 506.

The aspiration catheter 502, therefore, includes supply tube mounts or other structures that can be used to position a supply tube of an aspiration system to limit or prevent blockage of an aspiration lumen of an aspiration catheter during aspiration of thrombus, clots, or other aspirants. Positioning the supply tube mount in aspiration catheter 502 maintains the supply tube in a desired location against the wall of the aspiration catheter and so limits or prevents blockage of the aspiration lumen of the aspiration catheter 502 during aspiration of thrombus, clots, or other aspirants.

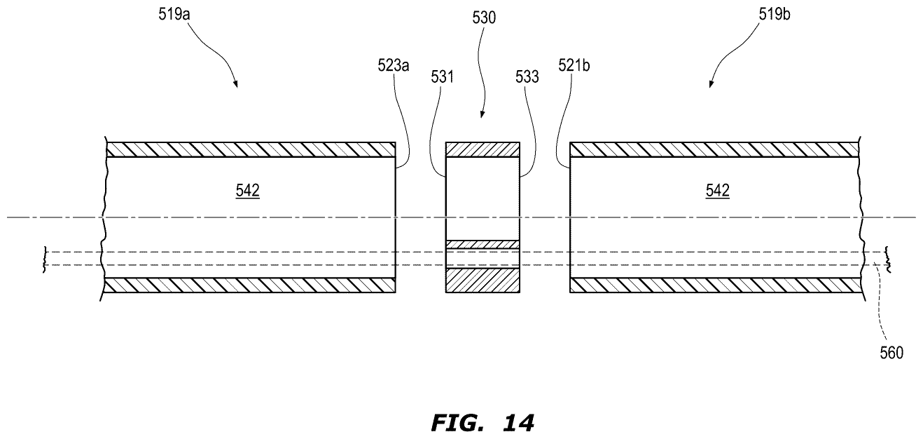

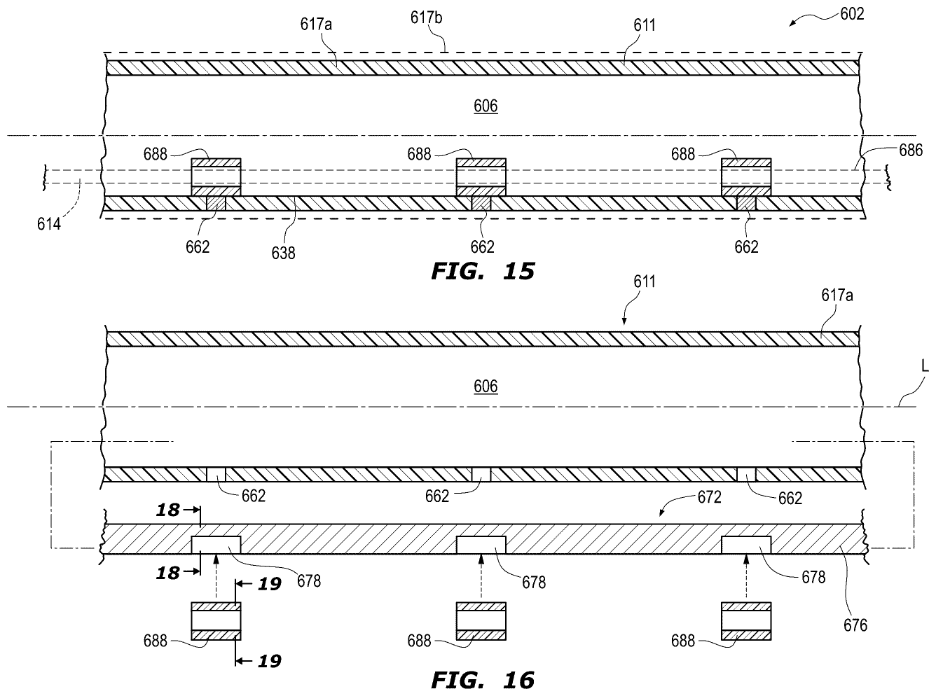

Turning to FIGS. 15-19, illustrated is another an aspiration catheter 602 that limits the possibility of inadvertent aspiration lumen blockage through entanglement between the supply tube and aspirants, including larger pieces of thrombus. The aspiration catheter 602 can be used with the systems 100 or 200 for aspirating thrombus. As such, the discussion relates to the aspiration catheter 102, 202, 302, 402, and 502 are also applicable and related to the aspiration catheter 602. Like reference numerals are used for like structure and/or elements with like functions. For simplicity of description, only a portion of the aspiration catheter 602 is depicted in FIGS. 15-19 with it understood that other portions of the aspiration catheter 602 can have the same or similar structures and functions to the any of the aspiration catheters 102, 202, 302, 402, or 502. For instance, the proximal ends, distal ends, and any other structures, etc. of the aspiration catheter, shaft, and supply tube, with associated jackets, etc. of any of the aspiration catheters 102, 202, 302, 402, or 502 can be included in aspiration catheter 602.

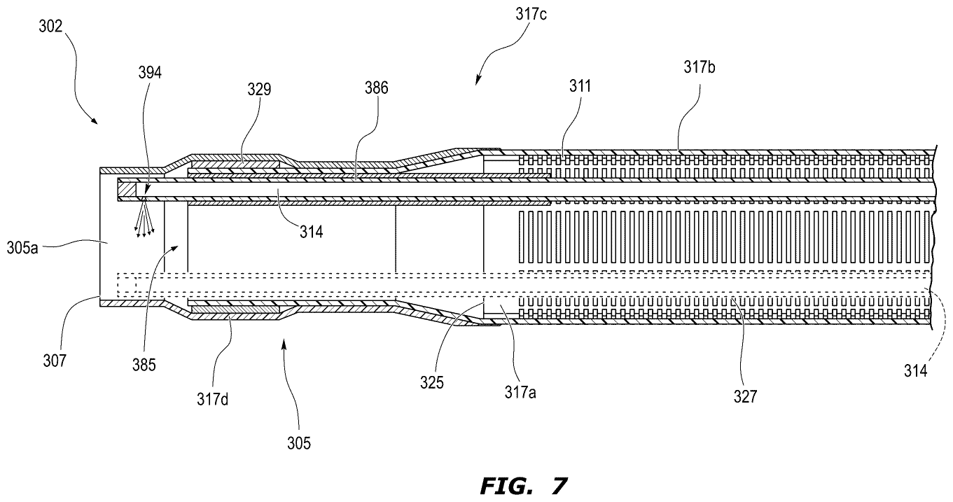

As illustrated in FIGS. 15-19, the aspiration catheter 602 includes an aspiration lumen 606 formed by a shaft 611 jacketed by a polymer jacket or a polymer jacket. For instance, a shaft body 617a is illustrated being jacketed by a jacket 617b, such as by heat-shrinking, laminating or otherwise applying the jacket 617b to the shaft body 617a. A supply tube 686 having the supply lumen 614 extends through the aspiration lumen 606 of the shaft body 617a towards the distal opening. The supply lumen 614 may be configured to provide a high pressure fluid injection or jet, such as saline, within the aspiration lumen 606 for macerating a thrombus as it is aspirated. The saline injection may occur through the orifice (such as orifice 494 (FIG. 9)) near the distal end of the supply lumen 614 if the opening of the supply lumen is plugged with a plug similar to plug 192 (FIG. 2). Aspiration catheter 602 may also include a radiopaque ring (such as radiopaque ring 329 (FIG. 7)) at or near the distal end of aspiration catheter 602 for identifying the location of aspiration. The radiopaque ring optionally encircles the aspiration catheter 602, with the radio ring being formed of any suitable radiopaque material, such as tantalum, tungsten, platinum/iridium, gold, silver, and combinations or modifications thereof.

The shaft 611 can be a tubular structure, such as a hypotube as described in relation to shafts 311 and 411 or a coiled structure as described in relation to shaft 511. Additionally, the shaft 611 can be formed from polymers, metals, alloys, braided structures, flat coiled structures, and combinations or modifications thereof. The shaft 611 can alternatively be a single or multi-lumen extruded polymer shaft. Furthermore, the jacket 617b can be formed of a variety of polymers and copolymers, plastics, PEBAX, HYTREL, rubber, nylon, polyethylene, polyurethane, polyester, polymer shrink tubing, such as polytetrafluoroethylene, fluorinated ethylene propylene, etc. and combinations or modifications thereof.

A plurality of supply tube mounts 688 can be disposed within the aspiration catheter 602 at various locations along a length of the aspiration catheter 602 or the shaft body 617a. The supply tube mounts 688 are welded or otherwise affixed, coupled or attached, to an inner surface 638 of the aspiration catheter 602, such as welding through shaft body 617a to the supply tube mount 688. Alternatively, a hole 662 can be formed in the shaft body 617a such as by laser cutting, drilling, etching, etc. and the supply tube mount 688 is welded to the shaft body 617a through the hole 662. For instance, a plug or slot weld can be used to couple, join, or attach the shaft body 617a and the supply tube mounts. Various other manners are possible of joining, coupling, or attaching the supply tube mount 688 to the shaft body 617a, such as via welding, laser welding, soldering, thermal bonding, chemical bonding, glues, adhesives, mechanical fasteners, combinations or modifications thereof.

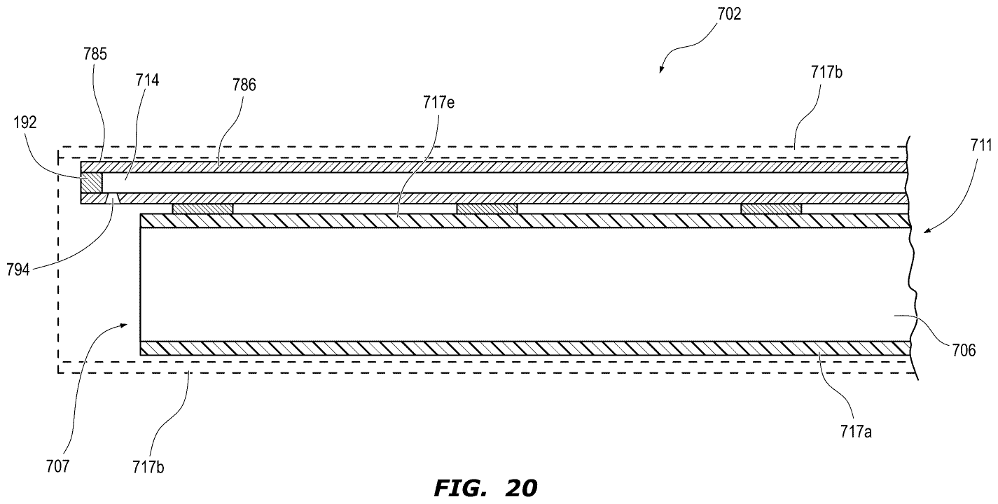

As illustrated in FIG. 15, the supply tube 686 is mounted or disposed towards one side of the aspiration lumen 606, thereby limiting the impact of the supply tube 686 upon flow of aspirant/thrombus along the aspiration lumen 606. In the illustrated configuration, the supply tube mounts 688 are fixed to the inner surface 638 of the aspiration lumen 606. The supply tube mount 688 includes, as illustrated in FIG. 19, a mount body 690 with a mount orifice 692 extending along a length of the mount body 690. The mount body 690 is joined, coupled or attached to the shaft body 617a as described herein. An inner surface 696 of the mount orifice 692 can be coated with a lubricous material to aid with movement of the supply tube 686 within the supply tube mounts 688 as the aspiration catheter 602 is advanced and moved within the tortuous anatomy of the patient. For instance, as the aspiration catheter 602 is turned or bends, the supply tube 686 can slide within the mount orifice 492. The lubricious material or coating can include silicone coatings, polymeric coatings, hydrophilic coatings, polytetrafluoroethylene (PTFE), or combinations or modifications thereof. Alternatively, the inner surface 696 of the mount orifice 692 can include a uniform or non-uniform structure that reduces frictional engagement between the supply tube 686 and the inner surface 696. In another configuration, a lubricious tube formed of polytetrafluoroethylene, fluorinated ethylene propylene, etc. can be pressed into the supply tube mount 688.

A length of each supply tube mount 688 can range from about 0.025 inches to about 0.5 inches, from about 0.03 inches to about 0.25 inches, from about 0.04 inches to about 0.25 inches, from about 0.05 inches to about 0.25 inches, about 0.1 inches, or is in a range between any two of the foregoing. A distance between two adjacent supply tube mounts 688 can range from about 1 inch to about 30 inches, from about 5 inches to about 25 inches, from about 10 inches to about 20 inches, from about 15 inches to about 20 inches. The distance between two adjacent supply tube mounts 688 can be uniform or non-uniform along the length of the aspiration catheter 602 or the shaft body 617a. For instance, a spacing between two adjacent supply tube mounts 688 can increase in a proximal-to-distal direction along the aspiration catheter 602 or the shaft body 617a to allow more movement of the supply tube 686 with the aspiration lumen 606. Alternatively, a spacing between two adjacent supply tube mounts 688 can decrease in a proximal-to-distal direction along the aspiration catheter 602 or the shaft body 617a to decrease movement of the supply tube 686 within the aspiration lumen 606.

To aid with positioning the supply tube mounts 688 within the aspiration lumen 606 in preparation for being joined, coupled, or attached to the inner surface 638, such as by welding, laser welding, soldering, adhesives, thermal bonding, etc., a positioning assembly 670 can support and apply a force to the supply tube mounts 688, such as illustrated in FIGS. 16-18. The positioning assembly 670 maintains a predefined separation between adjacent supply tube mounts 688 before being joined, coupled, or attached to the inner surface 638 and releasably retains the supply tube mounts 688 during manufacture of the aspiration catheter 602. For instance, the supply tube mounts 688 are releasably held by the positioning assembly 670 so they can be welded, laser welded, soldered, etc. to the inner surface 638 and become detached or released from the positioning assembly 670 by movement of the positioning assembly 670 in relation to the supply tube mounts 688 that are joined, coupled or attached to the inner surface 638, as will be described in more detail hereinafter.

With continued reference to FIGS. 16-18, the positioning assembly 670 includes a support mandrel 672 and an pusher 674 that cooperates with the support mandrel 672 to apply a force upon the support mandrel 672 to position the supply tube mounts 688 against the inner surface 638 of the shaft body 617a in preparation for being joined, coupled, or attached to the inner surface 638. Generally, the pusher 674 and the support mandrel 672 can have a length longer, short, or approximating a length of the aspiration catheter 602. As illustrated, the support mandrel 672 has an elongate body 676 with a shape and profile complementary to the aspiration lumen 606. Formed in the elongate body 676 are a plurality of recesses 678 that accept and retain the supply tube mounts 688 prior to being joined, coupled, or attached to the inner surface 638. With the supply tube mount 688 being generally cylindrical in form, for example, a surface of the recess 678 approximates a curvature of the supply tube mount 688. Frictional engagement between the inner surface of the recess 678 and the supply tube mount 688 retains the supply tube mount 688 within the recess 678 until the frictional engagement is overcome to release the supply tube mounts 688 during manufacture of the aspiration catheter 602. For instance, once the supply tube mount 688 is joined, coupled, or attached to the shaft body 617a, moving the support mandrel 672 in a direction transverse or perpendicular to a longitudinal axis L of the aspiration lumen 606 overcomes the friction-fit or interference connection between the supply tube mount 688 and the recess 678. Movement of the support mandrel 672 in a direction parallel to the longitudinal axis L, with or without movement in a direction transverse or perpendicular to a longitudinal axis L, can also release or overcome the frictional engagement between the supply tube mount 688 and the recess 678. The frictional engagement between the supply tube mount 688 and the recess 678 can be enhanced by providing surface coatings or treatments to one or both of the surface of the recess 678 and the supply tube mount 688 to increase the frictional engagement between the surface of the recess 678 and the supply tube mount 688. For instance, the coatings or surface treatment can include silicone coatings, polymeric coatings, hydrophilic coatings, polytetrafluoroethylene (PTFE), combinations or modifications thereof. Additionally, adhesives, glues, interference structures, detents, keys/keyways, combinations or modifications thereof. can also be used alone or with the coatings or surface treatments to increase the engagement between the surface of the recess 678 and the supply tube mount 688.

While reference is made to the supply tube mount 688 being generally cylindrical and the surface of the recess 678 having a complementary curvature to the supply tube mount 688, it will be understood that the supply tube mount 688 and the recess 678 can have other shapes or forms that are complementary or non-complementary so long as the recess 678 can retain the supply tube mount 688 to allow the supply tube mount 688 to be joined, coupled, or attached to the shaft body 617a. The retention of the supply tube mount 688 can be enhanced using adhesives, glues, interference structures, detents, keys/keyways, combinations or modifications thereof.

As mentioned above, the support mandrel 672 has an elongate body 676 having a shape and profile complementary to the aspiration lumen 606. The elongate body 676 is sized and configured to fit within the aspiration lumen 606 while leaving a gap 680 between the support mandrel 672 and the inner surface 638 of the shaft body 617a to accommodate the pusher 674. The gap 680 has a dimension D1 that is greater than a dimension D2 of the supply tube mount 688 retained within the recess 678. This allows the support mandrel 672 to have sufficient space to move and become released from the supply tube mount 688 and be removed from within the aspiration lumen 606.

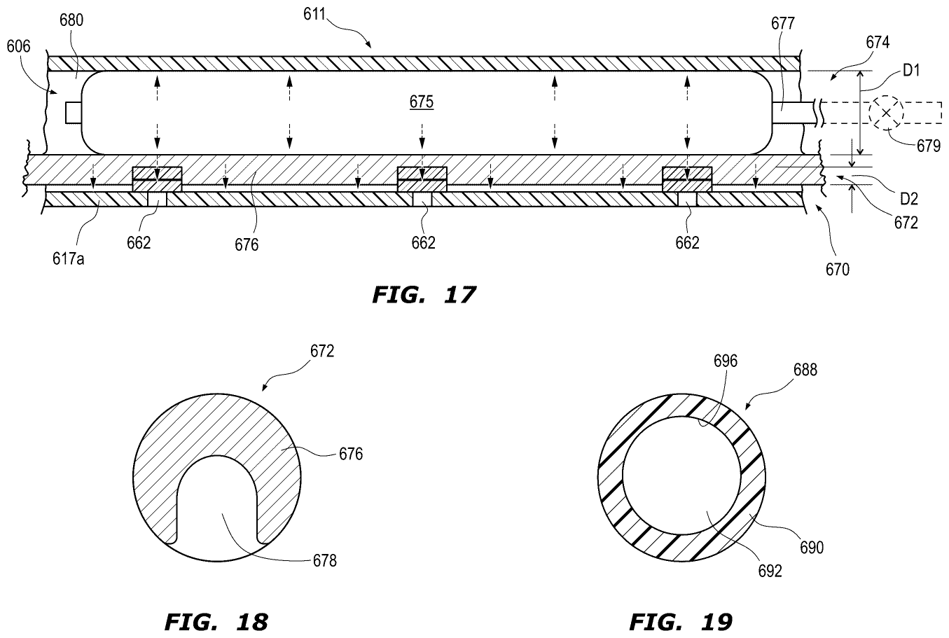

As illustrated in FIG. 17, the pusher 674 generates a force (illustrated by the arrows) on the support mandrel 672 to push the supply tube mounts 688 against the inner surface 638 of the aspiration lumen 606. The pusher 674 can generate and apply the force to the supply tube mounts 688 use structures, actuators, and mechanisms that are mechanical, pneumatic, hydraulic, electromechanical, or combinations or modifications. As illustrated, the pusher 674 is an expandable balloon having a balloon body 675 and an inflation tube 677 extending from the balloon body 675. The inflation tube 677 connects to an inflation source (not shown) such as a pump, syringe, reservoir, combinations or modifications thereof to provide a gas, liquid, or other fluid to inflate or expand the balloon body 675. A valve 679 associated with the inflation source or the inflation tube 677 can be used to control the flow gas, liquid, or other fluid to inflate, expand, deflate or collapse the balloon body 675.

While reference is made herein to the pusher 674 being a balloon or balloon catheter, it will be understood that in other configurations, an expandable frame or structure can be used to apply the pushing force, such expandable frame or structure being formed from shape memory material, alloys, metals, polymers, combinations or modifications thereof can be used to apply the pushing force. In still other configurations, threads, racks, pinions, screws, linkages, combinations or modifications therefor can be used to open or close a frame or structure to apply the force. It is also contemplated that combinations of any of the preceding structures, assemblies, or mechanisms can be used to apply the identified force. In still another configuration, the pusher 674 can be replaced with a ferro-magnetic rod that is advanced through the openings in the supply tube mounts 688 and one or more magnets, whether natural or electromagnetic, disposed on the outside of the catheter body 617a can pull the supply tube mounts 688 into contact with the catheter body 617a before attachment to the catheter body 617a as described herein. The ferro-magnetic rod can be optionally magnetic. In still another configuration, the support mandrel can be magnetic and/or include magnets within the recesses 678 to releasably hold the supply tube mounts 688.

Following positioning the supply tube mounts 688 into the recesses 678, as illustrated in FIG. 16, and with the pusher 674 disposed within the gap 680 and the supply tube mounts 688 positioned within the aspiration lumen 606, such as illustrated in FIG. 17, activating or actuating the pusher 674 to generate a force (illustrated by the arrows) on the support mandrel 672, the supply tube mounts 688 are pushed against the inner surface 638 of the aspiration lumen 606 over a region of the aspiration catheter 602. For instance, the valve 679 can be opened, the inflation source turned on to deliver gas, liquid, or other fluid to an interior of the balloon body 675 to inflate the balloon body 675, and the valve 679 closed to maintain the pushing force that places the supply tube mounts 688 in contact with the inner surface 638 for joining, coupling, or attaching the supply tube mounts 688 to the shaft body 617a through welding, laser welding, soldering, etc. With the supply tube mounts 688 in contact with the inner surface 638, plug or slot welding, laser welding, soldering, etc. is used to join, couple or attach the supply tube mounts 488 to the shaft body 617a.

Once the supply tube mounts 488 are joined, coupled or attached to the shaft body 617a, actuating the pusher 674 again decreases or eliminates the applied force sufficiently to withdraw the pusher 674 and the support mandrel 672. For instance, opening the valve 679 allows the gas, liquid, or other fluid to flow from the interior of the balloon body 675, such as through the inflation source drawing the gas, liquid, or other fluid to deflate the balloon body or venting the gas, liquid or fluid to atmosphere or a storage tank (not shown). This process is repeated along the length of the support mandrel 672 within the aspiration catheter 602 and/or along a length of the aspiration catheter 602 until the supply tube mounts 688 are fixed, attached or coupled to the shaft 611. For instance, when the support mandrel 672 has a length approximating a length of the aspiration catheter 602, the pusher 674 can be inflated/deflated in sequence over multiple regions as plug or slot welding, laser welding, soldering, etc. is used to join, couple or attach the supply tube mounts 488 to the shaft body 617a. When both the support mandrel 672 and the pusher 674 have lengths approximating the length of the aspiration catheter 602, the pusher 674 can be inflated over the length of the aspiration catheter 602 and then plug or slot welding, laser welding, soldering, etc. is used to join, couple or attach the supply tube mounts 688 to the shaft body 617a. In still another configuration, when the support mandrel 672 has a length shorter than the length of the aspiration catheter 602, the process of positioning the supply tube mounts 688 into the recesses 678, positioning the support mandrel 672 and the pusher 674 into the aspiration lumen 606, applying the force with the pusher 674, joining, coupling, and attaching the supply tube mount 688, and deflating or releasing the applied force exerted by the pusher 674 before removing the pusher 674 and the support mandrel 672 can be repeated along the length of the aspiration catheter 602 over multiple regions. Any combination of one or more of the steps of the proceeding process can be combined in any combination, order, etc.

The aspiration catheter 602 includes supply tube mounts or other structures that can be used to position a supply tube of an aspiration system to limit or prevent blockage of an aspiration lumen of an aspiration catheter during aspiration of thrombus, clots, or other aspirants. Positioning the supply tube mount in aspiration catheter 602 maintains the supply tube in a desired location against the wall of the aspiration catheter and so limits or prevents blockage of the aspiration lumen of the aspiration catheter 602 during aspiration of thrombus, clots, or other aspirants.

Turning to FIG. 20 illustrated is an aspiration catheter 702 that limits the possibility of inadvertent aspiration lumen blockage through entanglement between the supply tube and aspirants, including larger pieces of thrombus. The aspiration catheter 702 can be used with the systems 100 or 200 for aspirating thrombus. As such, the discussion relates to the aspiration catheter 102, 202, 302, 402, 502, and 602 are also applicable and related to the aspiration catheter 702. Like reference numerals are used for like structure and/or elements with like functions. For simplicity of description, only a portion of the aspiration catheter 702 is depicted in FIG. 20 with it understood that other portions of the aspiration catheter 702 can have the same or similar structures and functions to the any of the aspiration catheters 102, 202, 302, 402, 502, or 602. For instance, the proximal ends, distal ends, and any other structures, etc. of the aspiration catheter, shaft, and supply tube, with associated jackets, etc. of any of the aspiration catheters 102, 202, 302, 402, 502, or 602 can be included in aspiration catheter 702.

As illustrated in FIG. 20, aspiration catheter 702 includes an aspiration lumen 706 formed by a shaft 711, such as a hypotube. A supply tube 786 having the supply lumen 714 extends along an outer surface 717e of the shaft 711 towards and past a distal opening 707 of the shaft 711. The supply lumen 714 may be configured to provide a high pressure fluid injection or jet, such as saline, for macerating a thrombus as it is aspirated. The saline injection may occur through orifice 794 near the distal end of the supply lumen 714 if the opening of the supply lumen is plugged with a plug similar to plug 192 (FIG. 2). The supply tube 786 is joined, coupled, or attached to the outer surface 717e of the shaft 711, such as through welding, laser welding, soldering, thermal bonding, chemical bonding, glues, adhesives, mechanical fasteners, or combinations or modifications.

Disposed about the shaft 711 and the supply tube 786 is a jacket 717b. Unlike other aspiration catheters described herein, the jacket 717b surrounds not only a shaft body 717a of the shaft 711 but also the supply tube 786. The jacket 717b can be applied or mounted by heat-shrinking, or laminating for example. This jacket 717b seals the shaft 711 (including the one or more openings (see FIGS. 6-8) that increase a flexibility of shaft 711 to aid with advancement of the aspiration catheter 702 through the tortuous anatomy of a patient), but aids with mounting the supply tube 786 to the shaft 711. The jacket 717b extends distal of the distal end 785 of the supply tube 786 to form the distal opening 707 of the aspiration catheter 702 in a similar manner to aspiration catheter 302.

While reference is made to the shaft 711 being a hypotube, it will be understood that other tubular structures can be used for the shaft 711. For instance, the shaft 711 can be formed from polymers, metals, alloys, braided structures, flat coiled structures, and combinations or modifications thereof. Furthermore, the jacket 717b can be formed of a variety of polymers and copolymers, plastics, PEBAX, HYTREL, rubber, nylon, polyethylene, polyurethane, polyester, polymer shrink tubing, such as polytetrafluoroethylene, fluorinated ethylene propylene, etc. and combinations or modifications thereof.

The aspiration catheter 702, therefore, includes supply tube mounts or other structures that can be used to position a supply tube of an aspiration system to limit or prevent blockage of an aspiration lumen of an aspiration catheter during aspiration of thrombus, clots, or other aspirants. Positioning the supply tube mount in aspiration catheter 702 maintains the supply tube in a desired location against the wall of the aspiration catheter and so limits or prevents blockage of the aspiration lumen of the aspiration catheter 502 during aspiration of thrombus, clots, or other aspirants.

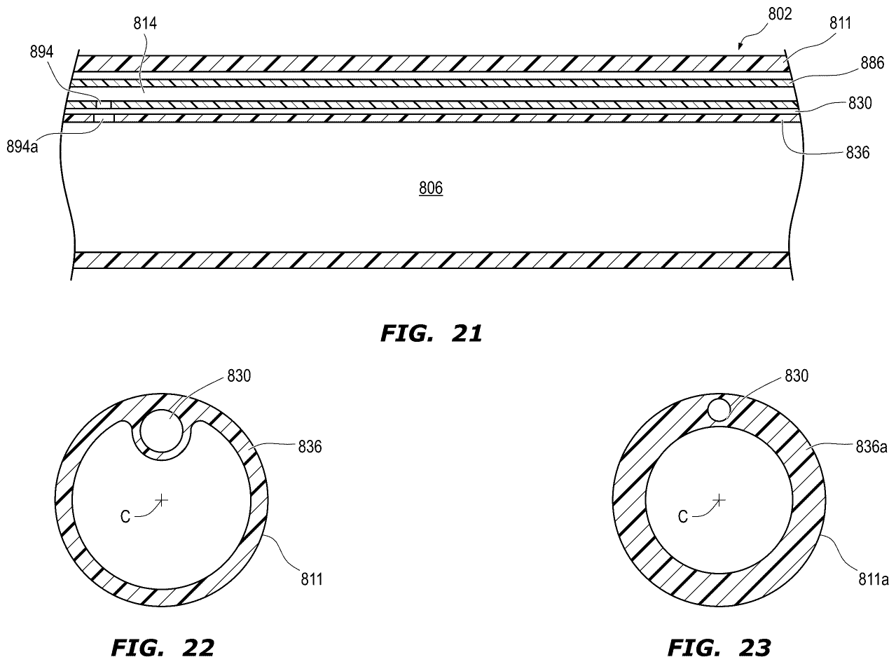

Turning to FIGS. 21-23 illustrated is an aspiration catheter 802 that limits the possibility of inadvertent aspiration lumen blockage through entanglement between the supply tube and aspirants, including larger pieces of thrombus. The aspiration catheter 802 can be used with the systems 100 or 200 for aspirating thrombus. As such, the discussion relates to the aspiration catheter 102, 202, 302, 402, 502, 602, and 702 are also applicable and related to the aspiration catheter 802. Like reference numerals are used for like structure and/or elements with like functions. For simplicity of description, only a portion of the aspiration catheter 802 is depicted in FIGS. 21-23 with it understood that other portions of the aspiration catheter 802 can have the same or similar structures and functions to the any of the aspiration catheters 102, 202, 302, 402, 502, 602, or 702. For instance, the proximal ends, distal ends, and any other structures, etc. of the aspiration catheter, shaft, and supply tube, with associated jackets, etc. of any of the aspiration catheters 102, 202, 302, 402, 502, 602 or 702 can be included in aspiration catheter 802.

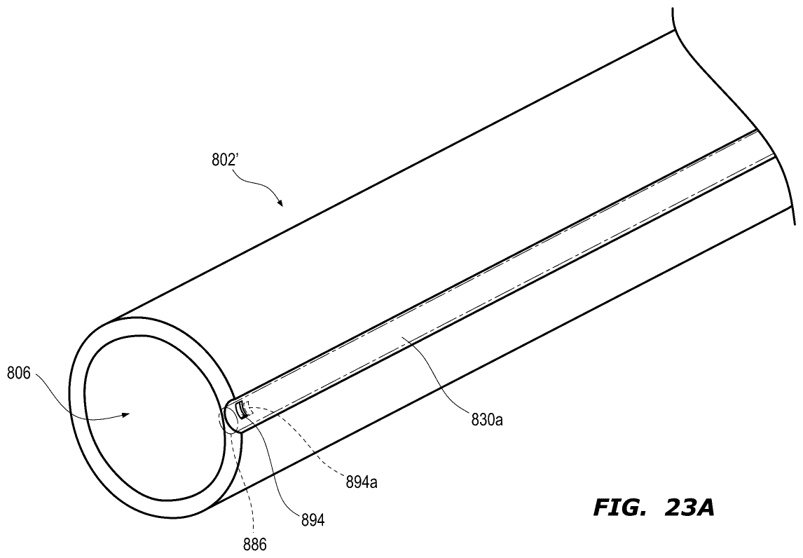

As illustrated in FIG. 21, the aspiration catheter 802 includes a shaft 811 possessing two lumens, such as an aspiration lumen 806 and a supply tube lumen 830 that receives a supply tube 886 with the supply lumen 814. The shaft 811 is a multi-lumen extruded tube, such as a tube formed of a polymer, PEBAX, VESTAMID, nylon, polyurethane, combinations or modifications thereof, and optionally reinforced with braids, wires, etc. The supply tube lumen 830 is formed in a wall 836 of the shaft 811. The wall 836 protrudes inwardly, optionally towards a center C of the aspiration lumen 806, to accommodate the supply tube lumen 830. However, as illustrated in FIG. 23, in other configurations, the wall 836a does not protrude inwardly towards the center to accommodate the supply tube lumen 830 in shaft 811a. Furthermore, the wall 836 can protrude towards another location different than the center C. Disposing the supply tube 886 within the supply tube lumen 830 limits the possibility of inadvertent aspiration lumen blockage through entanglement between the supply tube and aspirants, including larger pieces of thrombus.

The aspiration catheter 802, therefore, includes supply tube mounts or other structures that can be used to position a supply tube of an aspiration system to limit or prevent blockage of an aspiration lumen of an aspiration catheter during aspiration of thrombus, clots, or other aspirants. Positioning the supply tube mount in aspiration catheter 802 maintains the supply tube in a desired location against the wall of the aspiration catheter and so limits or prevents blockage of the aspiration lumen of the aspiration catheter 802 during aspiration of thrombus, clots, or other aspirants. As shown in FIG. 21, supply tube 886 includes an orifice 894 at or near a distal end of the supply lumen, which allows injection of pressurized fluid into the aspiration lumen 806, e.g., through an opening 894a in wall 836, which is aligned with orifice 894. In another embodiment, a distal portion of supply tube 886 may itself transition through the wall 836, so that the distal portion of the supply tube 886 is no longer within supply tube lumen 830, but is within aspiration lumen 806. In such an embodiment, the orifice at or near the distal end of the supply tube 886 jets directly into the aspiration lumen 806, without any need for an aligned opening 894a. FIG. 23A illustrates an additional embodiment, e.g., where the supply tube 886 may run within an exterior groove 830a of the aspiration catheter 802′, with orifice 894 aligned with opening 894a, to deliver a pressurized saline jet (or other fluid jet) into the aspiration lumen 806. Supply tube 886 may be secured within groove 830a using an adhesive, or any other mechanism, e.g., such as those described herein for securement.

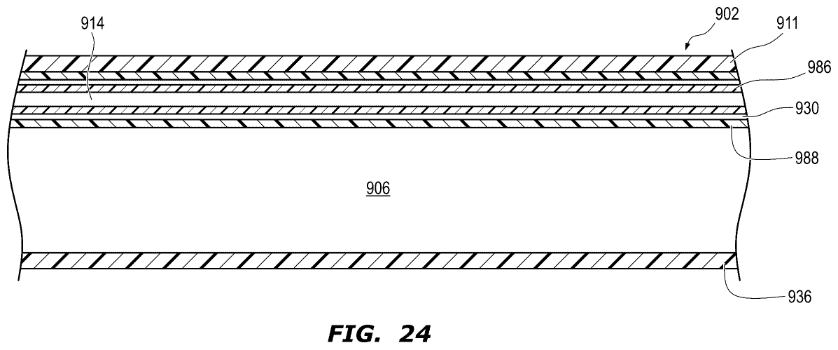

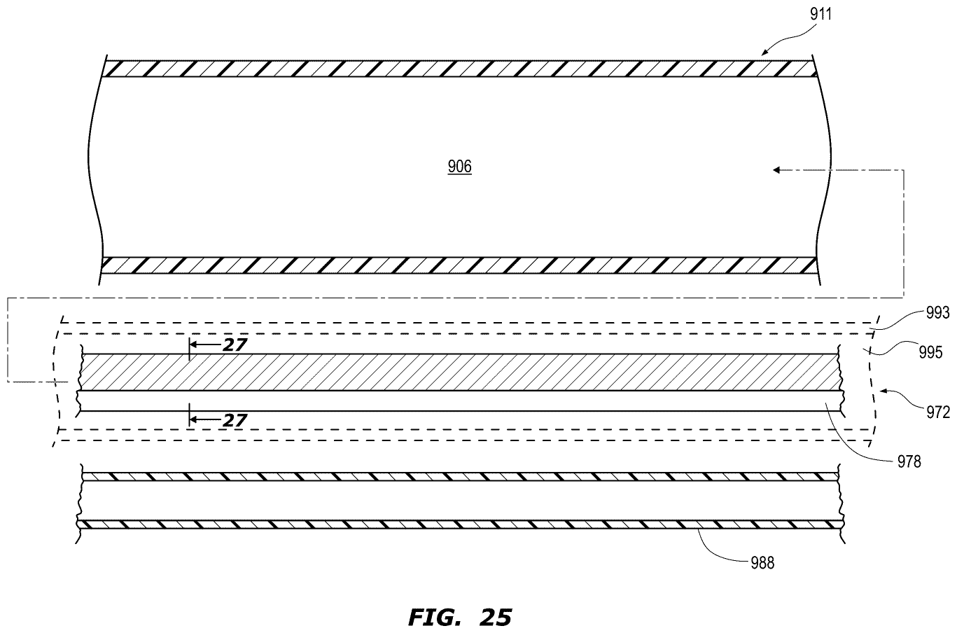

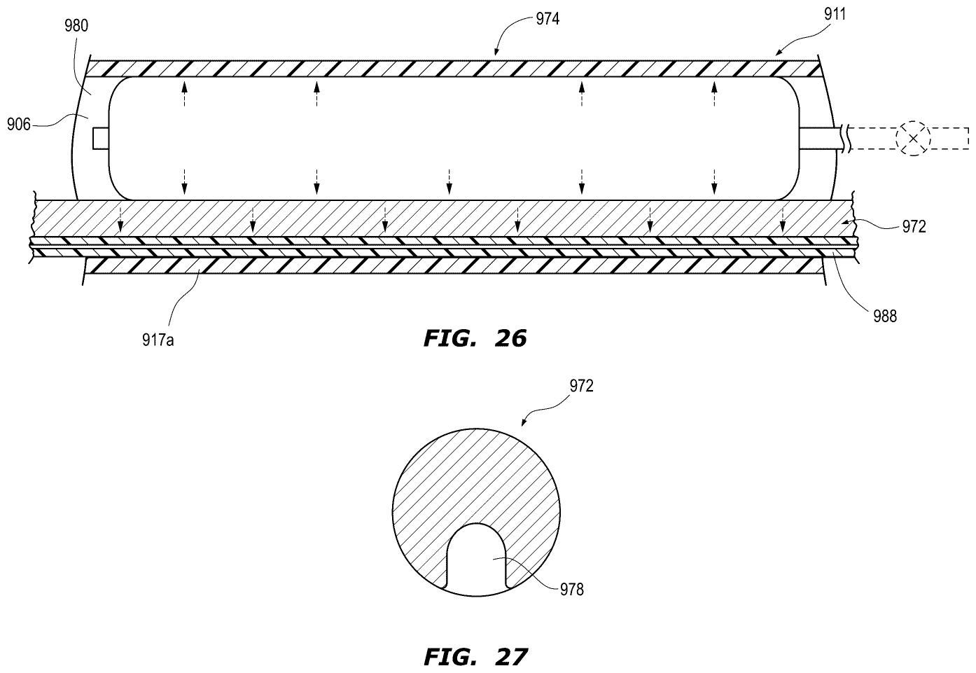

Turning to FIGS. 24-27 illustrated is another aspiration catheter 902 that limits the possibility of inadvertent aspiration lumen blockage through entanglement between the supply tube and aspirants, including larger pieces of thrombus. The aspiration catheter 902 can be used with the systems 100 or 200 for aspirating thrombus. As such, the discussion relates to the aspiration catheter 102, 202, 302, 402, 502, 602, 702, and 802 are also applicable and related to the aspiration catheter 902. Like reference numerals are used for like structure and/or elements with like functions. For simplicity of description, only a portion of the aspiration catheter 902 is depicted in FIGS. 24-27 with it understood that other portions of the aspiration catheter 902 can have the same or similar structures and functions to the any of the aspiration catheters 102, 202, 302, 402, 502, 602, 702, or 802. For instance, the proximal ends, distal ends, and any other structures, etc. of the aspiration catheter, shaft, and supply tube, with associated jackets, etc. of any of the aspiration catheters 102, 202, 302, 402, 502, 602, 702, or 802 can be included in aspiration catheter 902.

As illustrated in FIG. 24, the aspiration catheter 902 includes a shaft 911 possessing an aspiration lumen 906. Disposed within the aspiration lumen 906 is a supply tube lumen 930 that receives a supply tube 986 with the supply lumen 914. The supply tube lumen 930 is formed in an elongate supply tube mount 988 that is attached to a wall 936 of the shaft 911. The shaft 911 and the elongate supply tube mount 988 can be formed of a polymer, PEBAX, VESTAMID, nylon, polyurethane, combinations or modifications thereof, and optionally reinforced with braids, wires, etc.

The elongate supply tube mount 988 is an elongate tubular member having a length approximating a length of the aspiration catheter 902 or the shaft 911. The supply tube lumen 930 approximates an outer diameter of the supply tube 986 to frictionally hold the supply tube 986 within the supply tube lumen 930. The frictional contact between the supply tube 986 and the supply tube lumen 930 is sufficient to prevent unwanted movement of the supply tube 986 during delivery of pressurized jetted fluid during a procedure. Alternatively, the supply tube 986 can be fixed or coupled within the supply tube lumen 930 such as through glues, adhesives, thermal bonding, or combinations or modifications thereof.

The elongate supply tube mount 988 can be attached to the wall 936 during manufacture in a similar manner to that described in relation to aspiration catheter 602. For instance, instead of the support mandrel 672 of the positioning assembly 670 having the plurality of recesses 678, a support mandrel 972 includes an elongate channel or recess 978 extending along a length of the support mandrel 972. This elongate recess 978 receives the elongate supply tube mount 988. Following applying a glue or adhesive to the elongate supply tube mount 988, either before or after positioning within the elongate recess 978, the support mandrel 972 is disposed within the aspiration lumen 906. When the pusher 974 is positioned within the gap 980, and subsequently actuated (such as to inflate or otherwise apply a force upon the support mandrel 972), the pusher 974 applies a force upon the support mandrel 972 to position the elongate supply tube mount 988 against the inner surface 938 of the shaft body 917a. The glue or adhesive, for example, is then cured using ultraviolet radiation (UV) or allowed to cure over time. Once cured, the pusher 974 and support mandrel 972 can be removed.

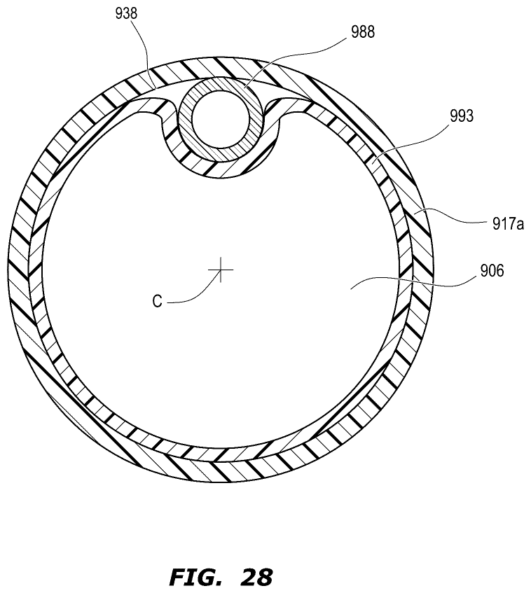

While reference is made to using glues or adhesives to attached the elongate supply tube mount 988, a laminate member 993 can also be used together with or as an alternate to the glues or adhesives (or other attachment, coupling or joining techniques or structures). For instance, as illustrated in FIGS. 25 and 28, the laminate member 993, such as a tubular member, can be positioned within the elongate channel or recess 978 before the elongate supply tube mount 988. The pusher 974 can be disposed within a laminate member lumen 995 of the laminate member 993 and when inflated the laminated member 993 is laminated against both the inner surface 938 of the shaft body 917a and also the elongate supply tube mount 988. Glues, adhesives, etc. can be applied to an outer surface of the laminate member 993 before being disposed within the aspiration lumen 906 and cured or allowed to cure as mentioned previously. Alternatively, the pusher 974 can apply heat to the laminate member 993 to aid with joining, coupling, or attaching the laminate member 993 to the shaft body 917a. For instance, the pusher 974 or another heater can be used to heat the laminate member 993, the elongate supply tube mount 988, and/or the shaft body 917a to create the bond between one or more of the laminate member 993, the elongate supply tube mount 988, and/or the shaft body 917a. Since the laminate member 993 is laminated against the inner surface of the shaft body 917a, the laminate member lumen 995 forms the aspiration lumen 906 of the aspiration catheter 902.

In still another configuration, while reference is made to the laminate member 993 being positioned within the elongate channel or recess 978 before the elongate supply tube mount 988, the laminate member 993 can be used directly with the supply tube 986 without the elongate supply tube mount 988. In such a case, the elongate recess 978 receives the laminate member 993 and the supply tube 986. Following applying a glue or adhesive to the supply tube 986 and/or the laminate member 993, either before or after positioning within the elongate recess 978, the support mandrel 972 is disposed within the aspiration lumen 906. When the pusher 974 is positioned within the gap 980, and subsequently actuated (such as to inflate or otherwise apply a force upon the support mandrel 972), the pusher 974 applies a force upon the support mandrel 972 to position the supply tube 986 and the laminate member 993 against the inner surface 938 of the shaft body 917a. The glue or adhesive, for example, is then cured using ultraviolet radiation (UV) or allowed to cure over time. Once cured, the pusher 974 and support mandrel 972 can be removed. When heat is used to attach the laminate member 993, the pusher 974 or another heater can be used to heat the laminate member 993, the supply tube 986, and/or the shaft body 917a to create the bond between one or more of the laminate member 993, the supply tube 986, and/or the shaft body 917a.

While FIGS. 25 and 28 illustrate the laminate member 993 circumferentially extending along the inner surface 938 of the shaft body 917a and covers or is in contact with a majority of the inner surface 938 excluding those portions of the inner surface 938 of the shaft body 917a in contact with the elongate supply tube mount 988 either directly or through glues, adhesives, thermal bonding, or combinations or modifications thereof. In other configurations, the laminate member 993 circumferentially extends along the inner surface 938 of the shaft body 917a and covers or is in contact with portions of the inner surface 938 sufficient to fix, couple, or mount the elongate supply tube mount 988 to the inner surface 938 of the shaft body 917a, i.e., less than a majority of the inner surface 938 of the shaft body 917a. Stated another way, the laminate member 993 is laminated onto the inner surface 938 of the shaft body 917a and secures the elongate supply tube mount 1088 within the shaft 1011.

The aspiration catheter 902, therefore, includes supply tube mounts or other structures that can be used to position a supply tube of an aspiration system to limit or prevent blockage of an aspiration lumen of an aspiration catheter during aspiration of thrombus, clots, or other aspirants. Positioning the supply tube mount in aspiration catheter 902 maintains the supply tube in a desired location against the wall of the aspiration catheter and so limits or prevents blockage of the aspiration lumen of the aspiration catheter 502 during aspiration of thrombus, clots, or other aspirants.

Although the systems for aspirating thrombus described herein are predominantly focused on aspiration, the systems may also, or alternatively, be configured for injecting or infusing fluids, with or without drugs, and may incorporate related features described in U.S. Pat. No. 10,716,583, issued Jul. 21, 2020, and entitled, “Systems and Methods for Removal of Blood and Thrombotic Material” and U.S. Pat. No. 10,492,805, issued Dec. 3, 2019, and entitled, “Systems and Methods for Thrombosis and Delivery of an Agent.” Each such patent is herein incorporated by reference in its entirety.

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

Following are some further example embodiments of the invention. These are presented only by way of example and are not intended to limit the scope of the invention in any way. Further, any example embodiment can be combined with one or more of the example embodiments.

Embodiment 1. An aspiration catheter comprising: an aspiration lumen configured to aspirate thrombus from a body lumen; a supply tube with a supply lumen configured to provide a high pressure fluid injection to the aspiration lumen; and a mount associated within the aspiration lumen and configured to position the supply lumen towards one side of the aspiration lumen to prevent blockage of the aspiration lumen through entanglement of thrombus with the supply tube.

Embodiment 2. The aspiration catheter of embodiment 1, wherein the mount comprises a mount body and a mount orifice, the mount orifice being configured to slidably receive the supply tube.

Embodiment 3. The aspiration catheter of any of the embodiments 1-2, wherein the mount orifice is coated with a lubricious coating.

Embodiment 4. The aspiration catheter of any of the embodiments 1-3, wherein the aspiration lumen is formed in a shaft, the shaft including a first shaft body segment and a second shaft body segment.

Embodiment 5. The aspiration catheter of any of the embodiments 1-4, wherein the mount comprises a supply tube mount disposed within the aspiration lumen and mounted to one of the first shaft body segment or the second shaft body segment, but not both the first shaft body segment and the second shaft body segment.

Embodiment 6. The aspiration catheter of any of the embodiments 1-5, wherein the supply tube mount aligns the first shaft body segment with the second shaft body segment for welding of the first shaft body segment to the second shaft body segment.

Embodiment 7. The aspiration catheter of any of the embodiments 1-6, wherein the supply tube mount extends from the first shaft body segment in a cantilevered fashion.

Embodiment 8. The aspiration catheter of any of the embodiments 1-7, further wherein the aspiration lumen is formed in a shaft, the shaft including a shaft body segment and a supply tube mount segment, the supply tube mount segment including a mount orifice configured to receive the supply tube.

Embodiment 9. The aspiration catheter of any of the embodiments 1-8, wherein a lumen of the shaft body segment and a lumen of the supply tube mount segment form the aspiration lumen.

Embodiment 10. The aspiration catheter of any of the embodiments 1-9, wherein the mount orifice is disposed within a wall of the supply tube mount segment.

Embodiment 11. The aspiration catheter of any of the embodiments 1-10, wherein a shaft forming the aspiration lumen is a laser cut hypotube laminated by a polymer.

Embodiment 12. The aspiration catheter of any of the embodiments 1-11, wherein a shaft forming the aspiration lumen is a coil laminated by a polymer.

Embodiment 13. The aspiration catheter of any of the embodiments 1-12, wherein a shaft forming the aspiration lumen is a multi-lumen extruded polymer shaft.

Embodiment 14. An aspiration catheter including: an elongate shaft configured for placement within a blood vessel; a supply lumen and an aspiration lumen each extending along the elongate shaft, the supply lumen having a proximal end and a distal end, and the aspiration lumen having a proximal end and a distal opening, the supply lumen being disposed externally to the aspiration lumen; and an orifice at or near the distal end of the supply lumen, the orifice being configured to allow injection of pressurized fluid into the aspiration lumen at or near the distal end of the aspiration lumen when the pressurized fluid is delivered through the supply lumen.

Embodiment 15. The aspiration catheter of embodiment 14, wherein the elongate shaft is a laser cut hypotube laminated by a polymer, the elongate shaft including the aspiration lumen.

Embodiment 16. The aspiration catheter of any of the embodiments 14-15, further including a supply tube forming the supply lumen, the supply tube mounted to an outer surface of the elongate shaft.

Embodiment 17. The aspiration catheter of any of the embodiments 14-16, further including a jacket surrounding the supply tube and the elongate shaft, the jacket extending distal a distal end of the supply tube.

Embodiment 18. The aspiration catheter of any of the embodiments 14-17, wherein the jacket is formed of a polymer.

Embodiment 19. The aspiration catheter of any of the embodiments 14-18, wherein the elongate shaft is a multi-lumen extruded polymer shaft.

Embodiment 20. The aspiration catheter of any of the embodiments 14-19, further including a radiopaque ring near the distal end of the aspiration catheter.

Embodiment 21. The aspiration catheter of any of the embodiments 14-19, further comprising an opening through a wall of the aspiration lumen aligned with the orifice of the supply lumen, which allows injection of pressurized fluid from the supply lumen through the orifice, through the opening, and into the aspiration lumen.

Embodiment 22. An aspiration catheter including: an elongate shaft configured for placement within a blood vessel, the elongate shaft having a distal end with an opening at the distal end; a supply lumen and an aspiration lumen each extending along the elongate shaft, the supply lumen having a proximal end and a distal end, and the aspiration lumen having a proximal end and a distal opening, the supply lumen being disposed within a supply tube and the aspiration lumen being formed from a shaft including a plurality of shaft body segments and a plurality of supply tube mount segments, each supply tube mount segment including a mount orifice configured to slidably receive the supply tube; and an orifice near the distal end of the supply lumen, the orifice being configured to allow injection of pressurized fluid into the aspiration lumen at or near the distal end of the aspiration lumen when the pressurized fluid is delivered through the supply lumen.

Embodiment 23. An aspiration catheter including: an elongate shaft configured for placement within a blood vessel, the elongate shaft having a distal end with an opening at the distal end; a supply lumen and an aspiration lumen each extending along an interior of the elongate shaft, coextensive with the elongate shaft, the supply lumen having a proximal end and a distal end, and the aspiration lumen having a proximal end and a distal opening, the supply lumen being disposed within a supply tube disposed within a plurality of mounts disposed within the aspiration lumen, the plurality of mounts being configured to position the supply lumen towards one side of the aspiration lumen to prevent blockage of the aspiration lumen through entanglement of thrombus with the supply tube; and an orifice near the distal end of the supply lumen, the orifice being configured to allow injection of pressurized fluid into the aspiration lumen at or near the distal end of the aspiration lumen when the pressurized fluid is pumped through the supply lumen.

Embodiment 24. The aspiration catheter of embodiment 23, further including a radiopaque ring encircling the aspiration catheter near the distal end of the aspiration catheter.

Embodiment 25. The aspiration catheter of any of the embodiments 23-24, wherein the elongate shaft is a laser cut hypotube laminated by a polymer jacket.

Embodiment 26. The aspiration catheter of any of the embodiments 23-25, wherein the elongate shaft is an extruded polymer shaft.

Embodiment 27. A system for aspirating thrombus, including: an aspiration catheter including: an elongate shaft configured for placement within a blood vessel of a subject; a supply lumen and an aspiration lumen each extending along an interior of the elongate shaft, the supply lumen being disposed within a supply tube disposed within a plurality of mounts disposed within the aspiration lumen, the plurality of mounts being configured to position the supply lumen towards one side of the aspiration lumen to prevent blockage of the aspiration lumen through entanglement of thrombus with the supply tube; and an orifice at or near a distal end of the supply lumen, the orifice being configured to allow injection of pressurized fluid into the aspiration lumen at or near the distal end of the aspiration lumen when the pressurized fluid is caused or allowed to flow through the supply lumen; a tubing set including a first lumen having a distal end configured to couple to the aspiration lumen of the aspiration catheter and a proximal end configured to couple to a vacuum source, and a second lumen having a distal end configured to couple to the supply lumen of the aspiration catheter and a proximal end configured to couple to a first fluid source; and a pressurization element configured to couple to the tubing set and further configured to pressurize fluid from the first fluid source or allow pressurized fluid from the first fluid source to be delivered to the supply lumen, such that the pressurized fluid is capable of flowing through the supply lumen from the proximal end of the supply lumen to the orifice at or near the distal end of the supply lumen.

Embodiment 28. A system for aspirating thrombus, including: an aspiration catheter including: an elongate shaft configured for placement within a blood vessel of a subject; a supply lumen and an aspiration lumen each extending along an interior of the elongate shaft, the supply lumen being disposed within a supply tube disposed within a plurality of mounts disposed within the aspiration lumen, the plurality of mounts being configured to position the supply lumen towards one side of the aspiration lumen to prevent blockage of the aspiration lumen through entanglement of thrombus with the supply tube; and an orifice at or near a distal end of the supply lumen, the orifice being configured to allow injection of pressurized fluid into the aspiration lumen at or near the distal end of the aspiration lumen when the pressurized fluid is caused or allowed to flow through the supply lumen; a tubing set including a first lumen having a distal end configured to couple to the aspiration lumen of the aspiration catheter and a proximal end configured to couple to a vacuum source, and a second lumen having a distal end configured to couple to the supply lumen of the aspiration catheter and a proximal end configured to couple to a first fluid source; and a pressurization element configured to couple to the tubing set and further configured to pressurize fluid from the first fluid source or allow pressurized fluid from the first fluid source to be delivered to the supply lumen, such that the pressurized fluid is capable of flowing through the supply lumen from the proximal end of the supply lumen to the orifice at or near the distal end of the supply lumen.

Embodiment 29. A method for forming an aspiration catheter, the method including: positioning a supply tube mount within a support mandrel; disposing the support mandrel within an aspiration lumen of a shaft body; applying a force from a pusher to the support mandrel to position the supply tube mount against an inner surface of the aspiration lumen; and following coupling the supply tube mount to the shaft body, removing the pusher and the support mandrel.

Embodiment 30. The method of embodiment 29, wherein coupling the supply tube mount to the shaft body comprises welding the supply tube mount to the shaft body.

Embodiment 31. The method of any of the embodiments 29-30, wherein welding the supply tube mount to the shaft body comprises plug welding the shaft body to the supply tube mount.

Embodiment 32. The method of any of the embodiments 29-31, wherein welding the supply tube mount to the shaft body comprises slot welding the shaft body to the supply tube mount to the shaft body.

Embodiment 33. The method of any of the embodiments 29-32, wherein positioning a supply tube mount within the support mandrel comprises positioning the supply tube mount within a recess of the support mandrel.

Embodiment 34. The method of any of the embodiments 29-33, wherein the supply tube mount frictionally engages with the recess.

Embodiment 35. The method of any of the embodiments 29-34, wherein disposing the support mandrel within the aspiration lumen of a shaft body further comprises aligning the supply tube mount with a hole formed in a wall of the shaft body.

Embodiment 36. The method of any of the embodiments 29-35, wherein disposing the support mandrel within the aspiration lumen of the shaft body, further comprises disposing the support mandrel within the aspiration lumen of the shaft body to form a gap between a wall of the shaft body and the support mandrel, the gap being configured to receive the pusher.

Embodiment 37. The method of any of the embodiments 29-36, wherein the pusher comprises a balloon body and wherein applying the force from the pusher to the support mandrel comprises inflating the balloon body.

Embodiment 38. The method of any of the embodiments 29-37, wherein the pusher comprises an expandable frame and wherein applying the force from the pusher to the support mandrel comprises expanding the expandable frame.

Embodiment 39. The method of any of the embodiments 29-38, wherein the expandable frame is formed of a shape memory material.

Embodiment 40. A method for forming an aspiration catheter, the method including: (a) positioning a first end of a supply tube mount into engagement with a first segment of a shaft body of the aspiration catheter; (b) positioning a second end of the supply tube mount into engagement with a second segment of a shaft body of the aspiration catheter; (c) following aligning the first segment and the second segment, coupling the first segment, the second segment, and the supply tube mount, and (d) repeating steps (a)-(c) until a length of the aspiration catheter is achieved.

Embodiment 41. The method of embodiment 40, wherein positioning a first end of a supply tube mount into engagement with a first segment of a shaft body of the aspiration catheter comprises fixing the first end of the supply tube mount to an inner surface of the first segment of the shaft body.

Embodiment 42. The method of any of the embodiments 40-41, wherein the first segment comprises a first end and a second end and the second segment comprises a third end and a fourth end.

Embodiment 43. The method of any of the embodiments 40-42, wherein coupling the first segment, the second segment, and the supply tube mount comprises fixing the second end of the first segment to the third end of the second segment, the supply tube mount being welded to one of the first segment or the second segment but not both the first segment and the second segment.

Embodiment 44. The method of any of the embodiments 40-43, wherein the first end of the supply tube mount is fixed to the first segment and a second end of the supply tube mount is free to move within a lumen of the second segment.

Embodiment 45. The method of any of the embodiments 40-44, wherein the first segment comprises one of a hypotube, a coil, or an extruded polymer tube.

Embodiment 46. The method of any of the embodiments 40-45, wherein aligning the first segment and the second segment comprises positioning the first segment and the second segment on a mandrel.

Embodiment 47. The method of any of the embodiments 40-46, wherein positioning the first end of the supply tube mount into engagement with the first segment of the shaft body of the aspiration catheter comprises fixing the first end of the supply tube mount to the second end of the first segment of the shaft body.

Embodiment 48. The method of any of the embodiments 40-47, wherein positioning the second end of the supply tube mount into engagement with the second segment of the shaft body of the aspiration catheter comprises fixing the second end of the supply tube mount to the third end of the second segment of the shaft body so that the supply tube mount is disposed between the first segment and the second segment.

Embodiment 49. A method for forming an aspiration catheter, the method including: positioning a supply tube upon an outer surface of a shaft body of an aspiration catheter to position an orifice of the supply tube distal a distal end of the shaft body, the orifice being in fluid communication with a supply lumen, the orifice being configured to allow injection of pressurized fluid; and mounting a jacket around the supply tube and the shaft body, a distal end of the jacket being distal the orifice, the jacket forming a distal opening of the aspiration catheter.

Embodiment 50. The method of embodiment 49, wherein the jacket is mounted by heat-shrinking or lamination.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. An aspiration catheter comprising:
an aspiration lumen configured to aspirate thrombus from a body lumen;
a supply tube with a supply lumen configured to provide a high pressure fluid injection to the aspiration lumen; and
a mount associated within the aspiration lumen and configured to position the supply lumen towards one side of the aspiration lumen to prevent blockage of the aspiration lumen through entanglement of thrombus with the supply tube.

2. The aspiration catheter of claim 1, wherein the mount comprises a mount body and a mount orifice, the mount orifice being configured to slidably receive the supply tube.

3. The aspiration catheter of claim 2, wherein the mount orifice is coated with a lubricious coating.

4. The aspiration catheter of claim 1, wherein the aspiration lumen is formed in a shaft, the shaft comprising a first shaft body segment and a second shaft body segment.

5. The aspiration catheter of claim 4, wherein the mount comprises a supply tube mount disposed within the aspiration lumen and mounted to one of the first shaft body segment or the second shaft body segment, but not both the first shaft body segment and the second shaft body segment.

6. The aspiration catheter of claim 5, wherein the supply tube mount aligns the first shaft body segment with the second shaft body segment for welding of the first shaft body segment to the second shaft body segment.

7. The aspiration catheter of claim 6, wherein the supply tube mount extends from the first shaft body segment in a cantilevered fashion.

8. The aspiration catheter of claim 1, further wherein the aspiration lumen is formed in a shaft, the shaft comprising a shaft body segment and a supply tube mount segment, the supply tube mount segment comprising a mount orifice configured to receive the supply tube.

9. The aspiration catheter of claim 8, wherein a lumen of the shaft body segment and a lumen of the supply tube mount segment form the aspiration lumen.

10. The aspiration catheter of claim 8, wherein the mount orifice is disposed within a wall of the supply tube mount segment.

11. The aspiration catheter of claim 1, wherein a shaft forming the aspiration lumen is a laser cut hypotube laminated by a polymer.

12. The aspiration catheter of claim 1, wherein a shaft forming the aspiration lumen is a coil laminated by a polymer.

13. The aspiration catheter of claim 1, wherein a shaft forming the aspiration lumen is a multi-lumen extruded polymer shaft.

14. An aspiration catheter comprising:
an elongate shaft configured for placement within a blood vessel;
a supply lumen and an aspiration lumen each extending along the elongate shaft, the supply lumen having a proximal end and a distal end, and the aspiration lumen having a proximal end and a distal opening, the supply lumen being disposed externally to the aspiration lumen; and
an orifice at or near the distal end of the supply lumen, the orifice being configured to allow injection of pressurized fluid into the aspiration lumen at or near the distal end of the aspiration lumen when the pressurized fluid is delivered through the supply lumen.

15. The aspiration catheter of claim 14, wherein the elongate shaft is a laser cut hypotube laminated by a polymer, the elongate shaft including the aspiration lumen.

16. The aspiration catheter of claim 15, further comprising a supply tube forming the supply lumen, the supply tube mounted to an outer surface of the elongate shaft.

17. The aspiration catheter of claim 16, further comprising a jacket surrounding the supply tube and the elongate shaft, the jacket extending distal a distal end of the supply tube.

18. The aspiration catheter of claim 17, wherein the jacket is formed of a polymer.

19. The aspiration catheter of claim 14, wherein the elongate shaft is a multi-lumen extruded polymer shaft.

20. The aspiration catheter of claim 14, further comprising a radiopaque ring near the distal end of the aspiration catheter.

21. The aspiration catheter of claim 14, further comprising an opening through a wall of the aspiration lumen aligned with the orifice of the supply lumen, which allows injection of pressurized fluid from the supply lumen through the orifice, through the opening, and into the aspiration lumen.

22. An aspiration catheter comprising:
an elongate shaft configured for placement within a blood vessel, the elongate shaft having a distal end with an opening at the distal end;
a supply lumen and an aspiration lumen each extending along the elongate shaft, the supply lumen having a proximal end and a distal end, and the aspiration lumen having a proximal end and a distal opening, the supply lumen being disposed within a supply tube and the aspiration lumen being formed from a shaft comprising a plurality of shaft body segments and a plurality of supply tube mount segments, each supply tube mount segment comprising a mount orifice configured to slidably receive the supply tube; and
an orifice near the distal end of the supply lumen, the orifice being configured to allow injection of pressurized fluid into the aspiration lumen at or near the distal end of the aspiration lumen when the pressurized fluid is delivered through the supply lumen.

23-50. (canceled)
